(19) United States
(12) Patent Application Publication
(10) Pub. No.: US 2012/0329666 A1
(43) Pub. Date: Dec. 27, 2012

(54) Peripheral Blood Biomarkers for Idiopathic Interstitial Pneumonia and Methods of Use

(75) Inventors: Mark P. Steele, Durham, NC (US); David A. Schwartz, Denver, CO (US)

(73) Assignee: Duke University, Durham, NC (US)

(21) Appl. No.: 13/500,595

(22) PCT Filed: Oct. 5, 2010
(86) PCT No.: PCT/US10/51496

§ 371 Date: Aug. 6, 2012

Related U.S. Application Data
(60) Provisional application No. 61/248,505, filed on Oct. 5, 2009.

(51) Int. Cl. C40B 30/04; G01N 33/566; C12Q 1/68

(52) U.S. Cl. 506/9; 435/6.12; 435/7.92; 436/501

(57) ABSTRACT

The present invention provides methods for diagnosing several types of diseases. Specifically, the present disclosure provides a panel of diagnostic genes, the differential expression of whose mRNAs or proteins in the sample of a subject indicates the presence of the disease in the subject. The methods involve extracting mRNAs or proteins from the sample and performing gene expression profiling assays such as microarray assay, RT-PCR oligonucleotide binding array, quantitative RT-PCR assay, proteomics assay, and/or ELISA assay.

### STATEMENT OF PRIORITY

This application claims the benefit, under 35 U.S.C. §119(e), of U.S. Provisional Application Ser. No. 61/248,505, filed Oct. 5, 2009, the entire contents of which are incorporated by reference herein.

### STATEMENT OF GOVERNMENT SUPPORT

This invention was produced in part using federal funds under NHLBI Grant Nos. HL095393 and HL099571. Accordingly, the U.S. Government has certain rights in this invention.

### FIELD OF THE INVENTION

The present disclosure relates generally to the field of medical diagnostics. In particular, the disclosure provides methods of prognosis of interstitial lung disease (ILD) and idiopathic interstitial pneumonia (IIP).

### BACKGROUND OF THE INVENTION

Interstitial lung disease (ILD), also known as diffuse parenchymal lung disease, refers to a group of lung diseases affecting the interstitium (King (2005) Am. J. Respir. Crit. Care Med. 172(3):268-279; Goldman et al. Cecil Medicine. 23^{rd} ed. Philadelphia, Pa.: Saunders (2008)). This group includes over 200 inflammatory and fibrosing disorders of the lower respiratory tract that affect primarily the alveolar wall structures as well as often involve the small airways and blood vessels of the lung parenchyma. Several causes of interstitial lung disease are known. They include occupational and environmental exposures, sarcoidosis, drugs, radiation, connective tissue or collagen diseases, genetic/familial predispositions, systemic sclerosis, scleroderma, rheumatoid arthritis and Lupus. When all known causes are ruled out, the condition is then called “idiopathic.”

Idiopathic interstitial pneumonias (IIPs) are interstitial lung diseases of unknown etiology that share similar clinical and radiologic features and are distinguished primarily by the histopathologic patterns on lung biopsy. In 2002, a consensus statement on the IIPs classified the interstitial pneumonias into distinct subtypes, based on a combination of clinical, radiographic, and pathologic criteria (Travis et al., (2002) American Thoracic Society/European Respiratory Society International Multidisciplinary Consensus Classification of the Idiopathic Interstitial Pneumonias This joint statement of the American Thoracic Society (ATS) and the European Respiratory Society (ERS) was adopted by the ATS Board of Directors, June 2001 and by the ERS Executive Committee, June 2001 Am. J. Respir. Crit. Care Med. 165(2):277-304). These subtypes include idiopathic pulmonary fibrosis/usual interstitial pneumonia (IPF/UIP), cryptogenic organizing pneumonia (COP), nonspecific interstitial pneumonia (NSIP), respiratory bronchiolitis-interstitial lung disease (RB-ILD), desquamative interstitial pneumonia (DIP), and histopathologic presentation; while some have a constellation of specific features that allows for a clear diagnosis to be made, all too frequently the type of IIP cannot be characterized.

The diagnosis of ILD, as well as the determination of the subtype of IIP, is challenging. In centers specializing in ILD, expert clinicians, radiologists, and pathologists interact in a multidisciplinary manner to review the tests to establish the correct diagnosis. However, expertise of this type is reasonably rare and community physicians are challenged in making these difficult diagnoses (Flaherty et al., (2004) Am. J. Respir. Crit. Care Med. 170:904-910). Moreover, inter-observer agreement among these professionals relating to ILD diagnosis is not consistently high. Even in the hands of academic clinicians, radiologists, and pathologists in tertiary care centers specializing in ILD, there remains significant inter-observer disagreement between professionals. The difficulty in making such diagnoses is most clinically relevant since the treatment approaches for the various subtypes are drastically different. Such disagreements therefore result in misdiagnosis and/or delayed treatment.

Therefore, less cumbersome and more accurate diagnostic approaches are needed to improve the accuracy of diagnosis of IIP and diagnose individuals at an earlier, more treatable, stage of their disease.

### SUMMARY OF THE INVENTION

The present invention provides a method of diagnosing interstitial lung disease in a subject or identifying a subject having an increased risk of developing interstitial lung disease, comprising: a) analyzing at least one biomarker in a sample from the subject; and b) comparing the analysis of (a) with an analysis of the at least one biomarker in individual samples from a group of mild interstitial lung disease subjects and/or a group of severe interstitial lung disease subjects, wherein an analysis of (a) that is similar to the analysis of (b) diagnoses interstitial lung disease in the subject or identifies the subject as having an increased risk of developing interstitial lung disease.

Also provided herein is a method of diagnosing interstitial lung disease in a subject or identifying a subject having an increased risk of developing interstitial lung disease, comprising: a) analyzing at least one biomarker in a sample from the subject; and b) comparing the analysis of (a) with an analysis of the at least one biomarker in individual samples from a group of control subjects, wherein an analysis of (a) that is different than the analysis of (b) diagnoses interstitial lung disease in the subject or identifies the subject as having an increased risk of developing interstitial lung disease.

Furthermore, the present invention provides a method of using biomarkers to diagnose or predict interstitial lung disease in a subject, comprising: a) analyzing at least one biomarker in a sample from a subject to create a gene expression profile; b) comparing the gene expression profile of (a) with a gene expression profile reference panel obtained from a group of mild interstitial lung disease subjects and/or a group of severe interstitial lung disease subjects; and c) identifying correlations between the gene expression profile of (a) and the gene expression reference panel of (b) that provide a diagnosis or prediction of interstitial lung disease in a subject, thereby using biomarkers to diagnose or predict interstitial lung disease in the subject.

The present invention further provides a method of using biomarkers to diagnose or predict interstitial lung disease in a subject, comprising: a) analyzing at least one biomarker in a sample from a subject to create a gene expression profile; b) comparing the gene expression profile of (a) with a gene expression profile reference panel obtained from a group of control subjects; and c) identifying differences between the gene expression profile of (a) and the gene expression reference panel of (b) that provide a diagnosis or prediction of interstitial lung disease n a subject, thereby using biomarkers to diagnose or predict interstitial lung disease in the subject.

In addition, the present invention provides a method of diagnosing or identifying increased risk of developing interstitial lung disease in a subject, comprising detecting at least one biomarker in a sample from the subject, wherein the detection of the at least one biomarker is correlated with a diagnosis or identification of increased risk of developing interstitial lung disease in the subject.

Further provided herein is a method of diagnosing interstitial lung disease in a subject or identifying a subject as having an increased risk of developing interstitial lung disease, comprising: a) quantifying the amount of at least one biomarker in a sample from the subject and comparing the amount of the at least one biomarker quantified in (a) with the amount of the at least one biomarker quantified in individual samples from a group of mild interstitial lung disease subjects and/or a group of severe interstitial lung disease subjects; and b) diagnosing interstitial lung disease in the subject or identifying the subject as having an increased risk of developing interstitial lung disease based on the comparison of the amount of the at least one biomarker of steps (a) and (b).

Further aspects of this invention include a method of diagnosing interstitial lung disease in a subject or identifying a subject as having an increased risk of developing interstitial lung disease, comprising: a) quantifying the amount of at least one biomarker in a sample from the subject; b) comparing the amount of the at least one biomarker quantified in (a) with the amount of the at least one biomarker quantified in individual samples from a group of control subjects; and c) diagnosing interstitial lung disease in the subject or identifying the subject as having an increased risk of developing interstitial lung disease based on the comparison of the amount of the at least one biomarker of steps (a) and (b).

Additionally provided herein is a method of identifying the effectiveness of interstitial lung disease treatment in a subject, comprising: a) quantifying the amount of at least one biomarker in a first sample taken from the subject prior to and/or at a defined first time point during interstitial lung disease treatment of the subject; b) quantifying the amount of the at least one biomarker of (a) in a second sample taken from the subject subsequent to and/or at a defined second time point later during interstitial lung disease treatment; and c) comparing the quantity of (a) with the quantity of (b), wherein a change in the quantity of (a) as compared with the quantity of (b) identifies the effectiveness of the interstitial lung disease treatment in the subject.

Also provided herein is a method of identifying the effectiveness of interstitial lung disease treatment in a subject, comprising: a) quantifying the amount of at least one biomarker in a first sample taken from the subject prior to and/or at a defined first time point during interstitial lung disease treatment of the subject; b) quantifying the amount of the at least one biomarker of (a) in a second sample taken from the subject subsequent to and/or at a defined second time point later during interstitial lung disease treatment; and c) comparing the quantity of (a) and the quantity of (b) with the quantity of the at least one biomarker in a gene expression reference panel obtained from a group of mild interstitial lung disease subjects and/or a group of severe interstitial lung disease subjects, wherein a change in the quantity of (a) and (b) as compared with the gene expression reference panel of (c) identifies the effectiveness of the interstitial lung disease treatment in the subject.

In further embodiments, the present invention provides a method of identifying the effectiveness of interstitial lung disease treatment in a subject, comprising: a) quantifying the amount of at least one biomarker in a first sample taken from the subject prior to and/or at a defined first time point during interstitial lung disease treatment of the subject; b) quantifying the amount of the at least one biomarker of (a) in a second sample taken from the subject subsequent to and/or at a defined second time point later during interstitial lung disease treatment; and c) comparing the quantity of (a) and the quantity of (b) with the quantity of the at least one biomarker in a gene expression reference panel obtained from a group of control subjects, wherein a change in the quantity of (a) and (b) as compared with the gene expression reference panel of (c) identifies the effectiveness of the interstitial lung disease treatment in the subject.

In the methods of this invention, the interstitial lung disease can be idiopathic interstitial pneumonia (IIP) and in some embodiments the IIP can be familial interstitial pneumonia (FIP).

Furthermore, in the methods of this invention described above, the biomarker of this invention can be one or more than one (e.g., 2, 3, 4, 5, 6, 7, 8, 9, 10, etc.) of any of the biomarkers of Table 2, any of the biomarkers of Table 3, any of the biomarkers of Table 4, any of the biomarkers of Table 5, any of the biomarkers of Table 12, any of the biomarkers of Table 13 and any combination thereof, either within a table and/or among these tables.

In additional embodiments of this invention, a method is provided of identifying a subject having an increased risk of developing severe interstitial lung disease, comprising: a) analyzing at least one biomarker in a sample from the subject; and b) comparing the analysis of (a) with an analysis of the at least one biomarker in samples from a group of control subjects, wherein an analysis of (a) that is different than the analysis of (b) identifies the subject as having an increased risk of developing severe interstitial lung disease. In embodiments of this method, the subject can have mild interstitial lung disease.

In the method above, the biomarker can be CAMP, CEACAM6, CTSG, DEFA3, DEFA4, OLFM4, HLTF and any combination thereof (Table 9) and the analysis of (a) that is different than the analysis of (b) can be an increase in an amount of the at least one biomarker in the sample from the subject relative to an amount of the at least one biomarker in the samples from the group of control subjects.

In further embodiments of the method above, the biomarker can be PACSIN1, FLJ11710, GABBR1, IGHM and any combination thereof (Table 9), and the analysis of (a) that is different than the analysis of (b) is a decrease in an amount of the at least one biomarker in the sample from the subject relative to an amount of the at least one biomarker in the samples from the group of control subjects.

In additional embodiments, the present invention provides a method of identifying a subject as having an increased risk of developing severe interstitial lung disease, comprising: a) quantifying the amount of at least one biomarker in a sample from the subject; b) comparing the amount of the at least one biomarker quantified in (a) with the amount of the at least one biomarker quantified in samples from a group of control subjects; and c) identifying the subject as having an increased risk of developing severe interstitial lung disease based on the comparison of the amount of the at least one biomarker of steps (a) and (b). In embodiments of this method the subject can have mild interstitial lung disease.

In the method above, the biomarker can be CAMP, CEACAM6, CTSG, DEFA3, DEFA4, OLFM4, HLTF and any combination thereof (Table 9) and the comparison of the amount of the at least one biomarker of steps (a) and (b) shows an increase in an amount of the at least one biomarker of step (a) relative to an amount of the at least one biomarker of step (b).

In further embodiments of the method above, the biomarker can be PACSIN1, FLJ11710, GABBR1, IGHM and any combination thereof (Table 9) and the comparison of the amount of the at least one biomarker of steps (a) and (b) shows a decrease in an amount of the at least one biomarker of step (a) relative to an amount of the at least one biomarker of step (b).

In the methods of this invention, the sample can be blood, bronchoalveolar lavage fluid, plasma, serum, sputum, tissue, cells and any combination thereof.

Further aspects of this invention include kits for diagnosing or identifying increased risk of developing interstitial lung disease in a subject, comprising an antibody that specifically binds a biomarker of this invention, a detection reagent, and instructions for use.

Also provided herein is a kit for diagnosing or identifying increased risk of developing interstitial lung disease in a subject, comprising a nucleic acid molecule that hybridizes with a biomarker of this invention, a detection reagent and instructions for use.

In the kits above, the biomarker to be detected can be any of the biomarkers of Table 2, any of the biomarkers of Table 3, any of the biomarkers of Table 4, any of the biomarkers of Table 5, any of the biomarkers of Table 12, any of the biomarkers of Table 13 and any combination thereof.

Additional aspects of this invention include kit for identifying increased risk of developing severe interstitial lung disease in a subject, comprising an antibody that specifically binds a biomarker of this invention (e.g., as listed in Table 9), a detection reagent, and instructions for use.

Further provided herein is a kit for identifying increased risk of developing severe interstitial lung disease in a subject, comprising a nucleic acid molecule that hybridizes with a biomarker of this invention (e.g., as listed in Table 9), a detection reagent and instructions for use.

The present invention provides peripheral blood biomarkers and/or biological signatures (e.g., gene or protein expression patterns) of idiopathic interstitial pneumonias, as well as methods of diagnosing IIPs using the provided peripheral blood biomarkers and/or biological signatures.

One aspect of the present invention provides a method of diagnosing or predicting the risk of interstitial lung disease comprising determining at least one biomarker in a sample of bodily fluid obtained from a subject and comparing the at least one biomarker obtained from a pre-symptomatic disease group and/or a symptomatic disease group.

Another aspect of the present invention provides a method of using peripheral blood biomarkers to diagnose or predict interstitial lung disease in a subject, comprising: (a) providing a sample of bodily fluid from a subject; (b) determining at least one biomarker from the sample to create a gene expression profile; (c) using the gene expression profile to compare with a gene expression profile reference panel; wherein the reference panel includes gene expression profiles obtained from pre-symptomatic and/or symptomatic interstitial lung disease groups.

Another aspect of the present invention provides a method for diagnosing or predicting interstitial lung disease in a subject, comprising: (a) obtaining a bodily fluid sample from the subject; and (b) detecting at least one biomarker in the sample, wherein the detecting of at least one biomarker is correlated with a diagnosis of interstitial lung disease.

Another aspect of the present invention provides a method of diagnosing a subject suspected of interstitial lung disease, comprising: (a) quantifying in a bodily fluid sample obtained from the subject the amount of at least one biomarker in a panel, the panel comprising at least one antibody and at least one antigen; (b) comparing the amount of the at least one biomarker quantified in the panel to a predetermined panel of biomarkers obtained from subjects having pre-symptomatic interstitial lung disease and symptomatic interstitial lung disease; and (c) determining whether the subject has a risk of interstitial lung disease based on the comparison of the biomarkers from steps (a) and (b).

Another aspect of the present invention provides a method for monitoring the effectiveness of interstitial lung disease treatment in a subject comprising: (a) obtaining a bodily fluid sample from a patient undergoing treatment for interstitial lung disease; (b) detecting the quantity of at least one biomarker to a reference panel, where the reference panel includes gene expression profiles obtained from pre-symptomatic and/or symptomatic interstitial lung groups; and (c) determining the effectiveness of the interstitial lung disease treatment.

In certain embodiments, the interstitial lung disease is idiopathic interstitial pneumonia (IIP). In other embodiments, the interstitial lung disease is familial interstitial pneumonia (IIP).

In some embodiments of this invention, the sample can be a bodily fluid. As used herein, the term “bodily fluid” refers to liquids that are inside the body of an animal, as well as fluids that are excreted or secreted from the body and body water that normally is not excreted or secreted. Such fluids include, but are not limited to, blood, bronchoalveolar lavage fluid, plasma, serum, and sputum. In one embodiment, the bodily fluid sample is selected from the group consisting of blood, bronchoalveolar lavage fluid, plasma, serum, and sputum. In certain embodiments, the bodily fluid is blood, preferably peripheral blood.

In other embodiments, the biomarker can be but is not limited to, surfactant protein-A, surfactant protein-D, MMP1, MMP8, IGFBP1, TNFRSF1, MALAT1, Annexin 1 (ANXA1), beta catenin (CTNNB1), and any combination thereof, along with the biomarkers as set forth in any of Tables 3, 4, 5, 9, 12 and 13. These markers can be employed in combination with any other biomarkers of this invention in the methods and kits described herein.

In some embodiments, the detecting comprises use of a microarray. In another embodiment, the detecting can be carried out with a quantitative RT-PCR oligonucleotide binding array, quantitative RT-PCR assay, proteomics assay, ELISA assay, immunoassay, hybridization assay, amplification assay and any combination thereof.

Another aspect of the present invention provides a kit for the diagnosing or predicting of interstitial lung disease in a subject, comprising an antibody and/or nucleic acid that specifically binds a biomarker of this invention, a detection reagent, and instructions for use. In certain embodiments, the kit further comprises at least one pre-fractionation spin column.

### DETAILED DESCRIPTION

For the purposes of promoting an understanding of the principles of the present invention, reference will now be made to particular embodiments and specific language will be used to describe the same. It will nevertheless be understood that no limitation of the scope of the disclosure is thereby intended, such alteration and further modifications of the invention as illustrated herein, being contemplated as would normally occur to one skilled in the art to which the invention relates.

Although the following terms are believed to be well understood by one of ordinary skill in the art, the following definitions are set forth to facilitate understanding of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art.

All patents, patent publications and non-patent publications referenced herein are incorporated by reference in their entireties.

As used herein, the terms “a” or “an” or “the” may refer to one or more than one. For example, “a” marker can mean one marker or a plurality of markers. Likewise, “a” cell can mean one cell of a plurality of cells.

As used herein, the term “and/or” refers to and encompasses any and all possible combinations of one or more of the associated listed items, as well as the lack of combinations when interpreted in the alternative (“or”).

As used herein, the term “about,” when used in reference to a measurable value such as an amount of mass, dose, time, temperature, and the like, is meant to encompass variations of 20%, 10%, 5%, 1%, 0.5%, or even 0.1% of the specified amount.

Unless otherwise defined, all technical twins used herein have the same meaning as commonly understood by one of ordinary skill in the art to which this disclosure belongs.

The present disclosure relates to methods for aiding in a diagnosis of, and methods for diagnosing, interstitial lung diseases. Biomarkers have been identified that may be utilized to aid in the diagnosis of and/or to diagnose interstitial lung diseases or to make a negative diagnosis. The biomarkers of this invention can also be employed in methods of identifying a subject at increased risk of developing an interstitial lung disease, in methods of distinguishing interstitial lung disease from other fibrotic lung diseases and in methods of determining the effectiveness of a treatment for interstitial lung disease. Such biomarkers are provided herein in Tables 2, 3, 4, 5, 12 and 13 and can be employed in the methods and kits of this invention in any combination among the listings on a given table and/or among the listings on different tables.

As used herein, the term “interstitial lung disease” (ILD) refers to a group of lung diseases affecting the interstitium, which includes over 200 inflammatory and fibrosing disorders of the lower respiratory tract that affect primarily the alveolar wall structures as well as often involve the small airways and blood vessels of the lung parenchyma.

As sued herein, the term “idiopathic interstitial pneumonias” (IIPs) refers to those interstitial lung diseases of unknown etiology that share similar clinical and radiologic features and are distinguished primarily by the histopathologic patterns on lung biopsy. IIPs may be classified into six (6) different subtypes, all of which are included within the scope of the present disclosure. These subtypes include idiopathic pulmonary fibrosis/usual interstitial pneumonia (IPF/UIP), cryptogenic organizing pneumonia (COP), nonspecific interstitial pneumonia (NSIP), respiratory bronchiolitis-interstitial lung disease (RB-ILD), desquamative interstitial pneumonia (DIP), and acute interstitial pneumonia (AIP). As used herein, the term “familial interstitial pneumonia” (FIP) refers to a form of interstitial pneumonia wherein at least two members of a family (related within three (3) degrees) have IIP. FIP can occur in families or sporadically, and is commonly characterized histologically by heterogeneous patches of fibrosis with excessive production and deposition of extracellular matrix components, such as collagen and fibronectin in the interstitial space.

As used herein, the term “subject” and “patient” are used interchangeably and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like.

As used herein, “analyzing” or “analysis” means detecting and/or quantifying one or more biomarker of this invention. In some embodiments, the detection and/or quantification is compared with detection and/or quantification of the biomarker(s) in a control sample(s) and in some embodiments the detection and/or quantification is compared with the detection and/or quantification of the biomarker(s) in reference sample(s) as described herein.

The methods of the present invention effectively differentiate between subjects with interstitial lung diseases (i.e., symptomatic or severe disease), pre-symptomatic (or mild disease) subjects with interstitial lung diseases, and normal subjects (i.e., control subjects). As defined herein, normal or control subjects are those individuals with a negative diagnosis with respect to interstitial lung diseases and/or without symptoms of interstitial lung disease. That is, normal or control subjects do not have or are not known or suspected to have interstitial lung disease.

The methods of this invention include detecting a biomarker in a sample from a subject. For example, biomarkers as listed in the tables herein have been identified that aid in the probable diagnosis of interstitial lung disease or aid in a negative diagnosis. In accordance with the present invention, at least one of the biomarkers is detected. In other embodiments, two or more, three or more, four or more, five or more, six or more, seven or more, eight or more, nine or more, ten or more, fifteen or more, twenty or more, thirty or more, forty or more, or fifty or more biomarkers, etc. can be detected and the presence or absence of such biomarkers can be correlated to a diagnosis of interstitial lung disease. As used herein, the term “detecting” includes determining the presence, the absence, the quantity, or a combination thereof, of any of the biomarkers of this invention.

In certain embodiments, selected groups of biomarkers find utility in the diagnosis of interstitial lung disease. For example, the presence of surfactant protein-A and surfactant protein-D correlates with survival and radiographic abnormalities in patients with familial idiopathic interstitial pneumonia. In other embodiments, the presence of MMP7, MMP1, MMP8, IGFBP1 and TNFRSF1 distinguishes IPF patients from controls.

As used herein, the term “biomarker” is defined as any molecule, such as a protein, peptide, protein fragment, nucleic acid molecule, polynucleotide and/or oligonucleotide, which is useful in differentiating interstitial lung disease samples from normal samples or differentiating mild interstitial lung disease from severe interstitial lung disease. The biomarker is typically differentially present or expressed in subjects having interstitial lung disease relative to normal subjects. However, some biomarkers, while not being differentially expressed between two classes may, nevertheless, be classified as a biomarker according to the present invention to the extent that they are significant in delineating subsets of groups in a classification group/tree. In Tables 2, 3, 4, 5, 9, 12 and 13 provided herein, the differential expression of the biomarkers of this invention is shown as a fold change, as compared with a normal control. Thus, the biomarkers of this invention are either present in a detectable amount as compared with a normal control that has no detectable amount of the biomarker and/or present in an amount that can be measured as a fold change (either an increase or decrease) as compared with a normal control. Thus, a differential expression pattern can be established for any combination of biomarkers of this invention on the basis of the values provided herein.

The differential expression, such as the over- or under-expression, of selected biomarkers relative to pre-symptomatic ILD subjects or normal subjects may be correlated to interstitial lung disease. By differentially expressed, it is meant herein that the biomarkers may be found at a greater or reduced level in one disease state compared to another, or that the biomarker(s) may be found at a higher frequency (e.g., intensity) in one or more disease states (e.g., pre-symptomatic ILD vs. ILD (i.e., symptomatic)).

The methods of this invention include detecting at least one biomarker. However, any number of biomarkers may be detected. It is preferred that at least two biomarkers are detected in the analysis. However, it is realized that three, four, or more, including all, of the biomarkers described herein may be utilized in the analysis. Thus, not only can one or more markers be detected, one to 60, preferably two to 60, two to 20, two to 10 biomarkers, two to 5 biomarkers, or some other combination, may be detected and analyzed as described herein. In addition, other biomarkers not herein described may be combined with any of the presently disclosed biomarkers to aid in the diagnosis of ILD. Moreover, any combination of the above biomarkers may be detected in accordance with the present invention.

The detection of the biomarkers described herein in a test sample may be performed in a variety of ways. In one embodiment, the method provides the reverse-transcription of complementary DNAs from mRNAs obtained from the sample. In such embodiments, fluorescent dye-labeled complementary RNAs are transcribed from complementary DNAs which are then hybridized to the arrays of oligonucleotide probes. The fluorescent color generated by hybridization is read by machine, such as an Agilent Scanner and data are obtained and processed using software, such as Agilent Feature Extraction Software (9.1).

As used herein, the term “gene expression profile” refers to the expression levels of mRNAs or proteins of a panel of genes in the subject. As used herein, the term “panel of diagnostic genes” refers to a panel of genes whose expression level can be relied on to diagnose or predict the status of the disease. Included in this panel of genes are those listed in Tables, 2, 3, 4, 5, 9, 12 and 13, as well as any combination thereof, as provided herein.

In other embodiments, complementary DNAs are reverse-transcribed from mRNAs obtained from the sample, amplified and simultaneously quantified by real-time PCR, thereby enabling both detection and quantification (as absolute number of copies or relative amount when normalized to DNA input or additional normalizing genes) of a specific gene product in the complementary DNA sample as well as the original mRNA sample.

In other embodiments of the present disclosure, the biomarkers of the present invention may also be detected, qualitatively or quantitatively, by immunoassay procedure. The immunoassay typically includes contacting a test sample with an antibody that specifically binds to or otherwise recognizes a biomarker, and detecting the presence of a complex of the antibody bound to the biomarker in the sample. The immunoassay procedure may be selected from a wide variety of immunoassay procedures known to the art involving recognition of antibody/antigen complexes, including enzyme-linked immunosorbent assays (ELISA), radioimmunoassay (RIA), and Western blots, and use of multiplex assays, including use of antibody arrays, wherein several desired antibodies are placed on a support, such as a glass bead or plate, and reacted or otherwise contacted with the test sample. Such assays are well-known to the skilled artisan and are described, for example, more thoroughly in Antibodies: A Laboratory Manual (1988) by Harlow & lane; Immunoassays: A Practical Approach, Oxford University press, Gosling, J. P. (ed.) (2001) and/or Current protocols in Molecular Biology (Ausubel et al.), which is regularly and periodically updated.

The antibodies to be used in the immunoassays described herein may be polyclonal antibodies and may be obtained by procedures well known to the skilled artisan, including injecting purified biomarkers into various animals and isolating the antibodies produced in the blood serum. The antibodies may alternatively be monoclonal antibodies whose method of production is well known to those skilled in the art, including injecting purified biomarkers into a mouse, for example; isolating the spleen cells producing the antiserum; fusing the cells with tumor cells to form hybridomas and screening the hybridomas. The biomarkers may first be purified by techniques similarly well known to the skilled artisan, including the chromatographic, electrophoretic and centrifugation techniques described previously herein. Such procedures may take advantage of the biomarker's size, charge, solubility, affinity for binding to selected components, combinations thereof, or other characteristics or properties of the protein. Such methods are known to the art and can be found, for example, in Current Protocols in Protein Science, J. Wiley and Sons, new York, N.Y., Coligan et al. (Eds.) (2002); Harris and Angal in Protein Purification Applications: A Practical Approach, Oxford University Press, New York, N.Y. (1990). Once the antibody is provided, a biomarker can be detected and/or quantitated by immunoassays as previously described herein and as are well known in the art.

Although specific procedures for immunoassays are well-known to the skilled artisan, generally, an immunoassay may be performed by initially obtaining a sample as previously described herein from a subject. The antibody may be fixed to a solid support prior to contacting the antibody with a test sample to facilitate washing and subsequent isolation of the antibody/biomarker complex. Examples of solid supports are well-known to the skilled artisan and include, for example, glass or plastic in the form of, for example, a microtiter plate. Antibodies can also be attached to the probe substrate, such as the ProteinChip® arrays.

After incubating the sample with the antibody, the mixture is washed and the antibody-marker complex may be detected. The detection can be accomplished by incubating the washed mixture with a detection reagent, and observing, for example, development of a color or other indicator. Any detectable label may be used. The detection reagent may be, for example, a second antibody that is attached to a detectable label. Exemplary detectable labels include magnetic beads (e.g., DYNABEADS™), fluorescent dyes, radiolabels, enzymes (e.g., horseradish peroxide, alkaline phosphatase and others commonly used in enzyme immunoassay procedures), and calorimetric labels such as colloidal gold, colored glass or plastic beads. Alternatively, the marker in the sample can be detected using an indirect assay, wherein, for example, a labeled antibody is used to detect the bound marker-specific antibody complex and/or in a competition or inhibition assay wherein, for example, a monoclonal antibody which binds to a distinct isotope of the biomarker is incubated simultaneously with the mixture. The amount of an antibody-marker complex can be determined by comparing to a standard, as would be well known in the art.

Throughout the assays, incubation and/or washing steps may be required after each combination of reagents. Incubation steps can vary from about 5 seconds to several hours, and in some embodiments, from about 5 minutes to about 24 hours. However, the incubation time will depend upon the particular immunoassay, biomarker, and assay conditions. Usually the assays will be carried out at ambient temperature, although they can be conducted over a range of temperatures, such as about 0° C. to about 40° C.

Kits are provided that may, for example, be utilized to detect the biomarkers described herein. The kits can, for example, be used to detect any one or more of the biomarkers described herein, which may advantageously be utilized for diagnosing or aiding in the diagnosis of ILD (pre-symptomatic or symptomatic), or in a negative diagnosis. For example, a kit may include an antibody that specifically binds to the marker and a detection reagent. Such kits can be prepared from the materials described herein. The kit may further include pre-fractionation spin columns as described herein, as well as instructions for suitable operating parameters in the form of a label or a separate insert.

The methods of the present disclosure have other applications as well. For example, the biomarkers can be used to screen for compounds that modulate the expression of the biomarkers in vitro or in vivo, which compounds in turn may be useful in treating or preventing ILD in subjects. In another example, the biomarkers can be used to monitor the response to treatments for ILD. In yet another example, the biomarkers can be used in heredity studies to determine if a subject is at risk for developing ILD.

Compounds suitable for therapeutic testing may be screened initially by identifying compounds that interact with one or more biomarkers of this invention. By way of example, screening might include recombinantly expressing a biomarker, purifying the biomarker, and affixing the biomarker to a substrate. Test compounds would then be contacted with the substrate, typically in aqueous conditions, and interactions between the test compound and the biomarker can be measured, for example, by measuring elution rates as a function of salt concentration. Certain proteins may recognize and cleave one or more biomarkers of this invention, in which case the proteins can be detected by monitoring the digestion of one or more biomarkers in a standard assay, e.g., by gel electrophoresis of the proteins.

In a related embodiment, the ability of a test compound to inhibit the activity of one or more of the biomarkers of this invention can be measured. One of skill in the art will recognize that the techniques used to measure the activity of a particular biomarker will vary depending on the function and properties of the biomarker. For example, an enzymatic activity of a biomarker may be assayed provided that an appropriate substrate is available and provided that the concentration of the substrate or the appearance of the reaction product is readily measurable. The ability of potentially therapeutic test compounds to inhibit or enhance the activity of a given biomarker can be determined by measuring the rates of catalysis in the presence or absence of the test compounds. The ability of a test compound to interfere with a non-enzymatic (e.g., structural) function or activity of one of the biomarkers listed herein can also be measured. For example, the self-assembly of a multi-protein complex which includes one of the biomarkers of this invention can be monitored by spectroscopy in the presence or absence of a test compound. Alternatively, if the biomarker is a non-enzymatic enhancer of transcription, test compounds which interfere with the ability of the biomarker to enhance transcription can be identified by measuring the levels of biomarker-dependent transcription in vivo or in vitro in the presence and absence of the test compound.

Test compounds that modulate the activity of any of the biomarkers of this invention can be administered to patients who have or who are at risk of developing interstitial lung disease(s). For example, the administration of a test compound that increases the activity of a particular biomarker may decrease the risk of ILD in a subject if the activity of the particular biomarker in vivo prevents the accumulation of proteins for ILD. Conversely, the administration of a test compound that decreases the activity of a particular biomarker may decrease the risk of ILD in a patient if the increased activity of the biomarker is responsible, at least in part, for the onset of ILD.

At the clinical level, screening a test compound includes obtaining samples from test subjects before and after the subjects are exposed to a test compound. The levels in the samples of one or more of the biomarkers of this invention may be measured and analyzed to determine whether the levels of the biomarkers change after exposure to a test compound. The samples may be analyzed by real-time PCR, as described herein, and/or the samples may be analyzed by any appropriate means known to one of skill in the art. For example, the levels of one or more of the biomarkers may be measured directly by Western blot using radio- or fluorescently-labeled antibodies that specifically bind to the biomarkers. Alternatively, changes in the levels of mRNA encoding the one or more biomarkers may be measured and correlated with the administration of a given test compound to a subject. In a further embodiment, changes in the level of expression of one or more of the biomarkers can be measured using in vitro methods and materials. For example, human tissue cultured cells that express, or are capable of expressing, one or more of the biomarkers of this invention can be contacted with a test compound or combination of test compounds. Subjects who have been treated with test compounds will be routinely examined for any physiological effects that may result from the treatment. In particular, the test compounds will be evaluated for the ability to decrease ILD likelihood in a subject. Alternatively, if the test compounds are administered to subjects who have previously been diagnosed with ILD, test compounds will be screened for the ability to slow or stop the progression of the disease.

### Materials and Methods

Study Population.

Within the cohort of patients with familial interstitial pneumonia, seven pre-symptomatic subjects (from seven different families) were identified with a high resolution computed tomography (HRCT) scan indicating a definite IPF pattern of disease, a self reported dyspnea score ≦1 (American Thoracic Society dyspnea scale), and an average % predicted DLCO (diffusing capacity of carbon monoxide) of ≧79.3±12.4 as representative for the pre-symptomatic disease group. Seven symptomatic patients with FIP (form seven different families) with a definite IPF HRCT pattern of disease were also identified. Symptomatic disease was defined as dyspnea score ≧4 and an average % predicted DLCO≦39.4±10.8. Medical histories were obtained to eliminate patients exposed to fibrosing agents (e.g., asbestos) or medical treatments (e.g., Bleomycin). Subjects with systemic connective tissue or inflammatory diseases (e.g., rheumatoid arthritis), diabetes mellitus, atherosclerosis or current administration of corticosteroids or immunosuppressive drugs were also excluded from this study. Final FIP diagnosis in the symptomatic disease group was made by a surgical lung biopsy. Healthy controls (N=11) were selected based on the absence of any family history or current symptoms of lung disease. The average age in the pre-symptomatic disease group is approximately 64 years, while the average age in the symptomatic disease and control group is approximately 59 years. The clinical and demographic variables are summarized in Table 1.

As shown in Table 1, peripheral blood gene expression profiles were generated from patients with pre-symptomatic disease (no dyspnea with normal DLCO) or symptomatic pulmonary fibrosis (dyspnea with DLCO<60%), and these profiles were compared to age and gender matched non-diseased, healthy controls. Within the cohort of familial interstitial pneumonia patients, by screening unaffected family members, 66 pre-symptomatic subjects with some form of IIP were identified. Of these 66 pre-symptomatic individuals, seven met study criteria consisting of: 1) a consensus diagnosis of probable or definite disease; 2) a self reported dyspnea score ≦1 (American Thoracic Society dyspnea scale: either no dyspnea or dyspnea walking up a hill); 3) a DLCO (diffusing capacity of carbon monoxide) of ≧70% predicted; 4) a medical history that eliminated patients with secondary causes of pulmonary fibrosis such as environmental or drug exposure, systemic disease, or other causes of pulmonary fibrosis; and 5) no current administration of corticosteroids, immunosuppressive drugs, hormone therapy (e.g., estrogens or progestins), insulin, or other drugs likely to influence the peripheral blood transcriptome. Symptomatic disease subjects were selected based on a consensus diagnosis of probable or definite disease with a dyspnea score ≧4 and an average % predicted DLCO≦39.4±10, and patients were similarly excluded as outlined in items 4 and 5 as above.

Blood Collection.

Peripheral blood was collected from FIP patients, and age and gender matched healthy normal controls, as approved by the corresponding human subjects review board. All subjects gave informed consent. Subjects participating in the study were instructed to fast eight hours prior to blood collection in the early morning (7-9 AM). Subjects were also instructed to refrain from taking medications before the morning of blood collection. Approximately 2.5 ml of whole blood was collected in PAXgene™ Blood RNA tubes (Qiagen, Valencia, Calif.).

RNA isolation and Microarray Analysis.

RNA was isolated using the PAXgene™ Blood RNA kit (Qiagen, Valencia, Calif.) according to the manufacturer's instructions. RNA from replicate tubes was pooled and the concentration determined using the Ribo-Green RNA Quantification kit (Molecular Probes, Eugene, Oreg., USA). The quality of total RNA was analyzed using the RNA 6000 Nano Labchip kit on a 2100 BioAnalyzer (Agilent Technologies, Santa Clara, Calif.). Gene expression analysis was conducted using Agilent Whole Human Genome 4×44 multiplex format oligo arrays (Agilent Technologies) following the Agilent single-color microarray-based gene expression analysis protocol. This array contains 43,376 biological features with 41,000 unique probes with annotations derived from the Golden path Ensemble Unigene Human genome build 33. Starting with 500 ng of total RNA, Cy3 labeled cRNA was produced according to the manufacturer's protocol. For each sample, 1.65 ug of Cy3 labeled eRNAs were fragmented and hybridized for 17 hours in a rotating hybridization oven. Slides were washed and then scanned with an Agilent Scanner. All arrays were run in the same micro array core facility. Data were obtained using the Agilent Feature Extraction software (9.1), using the 1-color defaults for all parameters. This software was also used to perform error modeling, adjusting for additive and multiplicative noise. The resulting data were processed using the Rosetta Resolver® system version 7.0 (Rosetta Biosoftware, Kirkland, Wash.). The signals produced by feature extraction were converted to log 2 values (base 2 log scale) and transformed according to the “quantile normalization.” Statistical comparisons were done using the R version of MAANOVA as described by Gary A. Churchill (http://researchjax.org/faculty/churchill/index.html). The F2 statistics were applied to quantify the strength of associations. Significance levels (p-values) were determined based on permutation analysis with 500 permutations. All the data files (GSE11720) are posted at the GEO website (http://ncbi/geo/).

Gene Ontology and Functional Network Analysis.

Data were analyzed through the use of Ingenuity Pathways Analysis (Ingenuity Systems®, www.ingenuity.com). Ingenuity Pathway Analysis (IPA) is a web-based application that enables the visualization, discovery and analysis of molecular interaction networks within gene expression profiles. All generated gene lists and corresponding expression levels, represented as the log 2 ratios, were uploaded within the IPA database for further analysis. Both gene symbols and GenBank® database accession numbers were used with no apparent differences in results. These genes, called focus genes, were overlaid onto a global molecular network developed from information contained in the Ingenuity knowledge base. The IPA knowledge base represents a proprietary ontology of over 600,000 classes of biologic objects spanning genes, proteins, cells and cell components, anatomy, molecular and cellular processes, and small molecules. Networks of the focus genes were then algorithmically generated based on their connectivity. The Functional Analysis of a network identified the biological functions and/or diseases that were most significant to the genes in the network. The network genes associated with biological functions and/or diseases in the Ingenuity knowledge base were considered for the analysis. Fischer's exact test was used to calculate a P-value determining the probability that each biological function and/or disease assigned to that network is due to chance alone. Canonical Pathways Analysis identified the pathways from the Ingenuity Pathways Analysis library of canonical pathways that were most significant to the dataset. The significance of the association between the dataset and the canonical pathway was measured in two ways: 1) a ratio of the number of genes from the dataset that map to the pathway divided by the total number of molecules that exist in the canonical pathway is displayed, and 2) Fischer's exact test was used to calculate a P-value. Biomarker Analysis allows the identification of the most relevant molecular biomarker candidates from a dataset based on contextual information such as mechanistic association with a disease or detection in bodily fluids.

### EXAMPLES

### Example 1

### Pre-Symptomatic and Symptomatic Disease Comparison

Testing was done to determine whether peripheral blood gene expression profiles could be used to distinguish pre-symptomatic and symptomatic disease. These disease groups consisted of seven samples each. The generated expression profiles were analyzed using the Rosetta Resolver system. This analysis revealed only 69 significantly changed probes of which eight are unknown. Additional cluster analysis revealed that this subset of probes was not sufficient to distinguish both groups. This implies that the expression levels of pre-symptomatic and symptomatic disease, as tested with Agilent whole human genome oligo-micro arrays, did not change strongly enough to allow a statistically significant separation between pre-symptomatic and symptomatic disease in a small sample size study.

### Example 2

### A Molecular Signature in Lung Differentiates Sporadic from familial interstitial pneumonia

To develop a molecular signature of sporadic and familial interstitial pneumonia in lung tissue, a dataset was generated and analyzed by using Agilent Whole Genome oligonucleotide microarrays utilizing RNA extracted from surgical lung biopsy samples. The dataset was analyzed by statistical analysis of microarray (SAM) using a false discovery rate of <5%, and 138 differentially expressed transcripts with >1.8-fold change were identified. While one sporadic case clustered with controls, disease and control could be distinguished. In general, patients with sporadic or familial disease are more readily distinguished compared to the histopathology of usual interstitial pneumonia (UIP) or nonspecific interstitial pneumonia (NSIP). This study demonstrates that specific molecular signatures can be identified in sporadic and familial interstitial pneumonias, and the histologic subtypes of IIP.

### Example 3

### Molecular Signatures in Peripheral Blood are Predictive of Diagnosis Idiopathic Pulmonary Fibrosis (IPF)

To develop a molecular signature of the presence of IPF in peripheral blood, peripheral blood gene expression profiles were generated using Agilent Whole Human Genome oligonucleotide-microarrays from patients with pre-symptomatic disease (no dyspnea with normal DLCO) or symptomatic pulmonary fibrosis (dyspnea with DLCO<60%), and these profiles were compared to age and gender matched non-diseased, healthy controls (Table 1). Within the cohort of familial interstitial pneumonia patients, by screening unaffected family members, 66 pre-symptomatic subjects with some form of IIP were identified. Of these 66 pre-symptomatic individuals, seven were identified that met study criteria consisting of 1) a consensus diagnosis of probable or definite disease, 2) a self reported dyspnea score ≦1 American Thoracic Society dyspnea scale: either no dyspnea or dyspnea walking up a hill), 3) a DLCO (diffusing capacity of carbon monoxide) of ≧70% predicted, 4) a medical history that eliminated patients with secondary causes of pulmonary fibrosis such as environmental or drug exposure, systemic disease, or other causes of pulmonary fibrosis, and 5) no current administration of corticosteroids, immunosuppressive drugs, hormone therapy (e.g., estrogens or progestins), insulin, or other drugs likely to influence the peripheral blood transcriptome. Symptomatic disease subjects were selected based on a consensus diagnosis of probable or definite disease with a dyspnea score ≧4 and an average % predicted DLCO≦39.4±10, and patients were similarly excluded as outlined in items 4 and 5 as above.

### Example 4

### A Peripheral Blood Molecular Signature for FIP

Although a gene expression pattern that distinguished pre-symptomatic from symptomatic disease could not be derived, it was reasoned that candidate biomarkers for pre-symptomatic and symptomatic disease could be revealed by comparing the profiles from each individual disease group with the profiles from normal healthy controls. A cut off P-value of ≦0.001 was applied using the Rosetta system for each group comparison with the healthy normal control group. In this way, 286 and 406 differentially expressed probes for the pre-symptomatic and symptomatic disease group, respectively, were identified, with 36 probes in common. Next, all ambiguous probes (unknown or partial sequences in the genome) were removed, resulting respectively in 214 and 267 specific genes for pre-symptomatic (Table 2) and symptomatic disease (Table 3).

From these genes, probes were selected with a fold difference of at least 1.5, reducing the list of genes to 125 for the pre-symptomatic disease group and 216 for the symptomatic disease group. These 341 genes were subsequently used for cluster analysis. A heat map shows that these 341 genes (selected from the individual group comparisons of pre-symptomatic and symptomatic with healthy normal control group) are not sufficient to separate pre-symptomatic from symptomatic disease, corroborating the initial analysis between the two disease stages. However, the cluster analysis based on these 341 genes demonstrates a clear distinction between the normal controls and the diseased population (pre-symptomatic or symptomatic disease) (Student T-test P-values between 3.2 E-7 and 1.4 E-21), suggesting that a peripheral blood expression signature for presymptomatic or symptomatic forms of FIP is feasible.

### Example 5

### Functional Analysis of Differentially Expressed Genes

The functional analysis tool of the Ingenuity Pathway Analysis (IPA) software associates biological functions and diseases to the experimental results and calculates a significance value that is a measure of the likelihood that the association between a set of genes and a given process is due to random chance. Based on the two comparisons between IPF (pre-symptomatic and symptomatic) versus normal, the list of 214 (Table 2) genes and 267 (Table 3) genes was subjected to a functional dataset analysis. The results show that the distinction between the pre-symptomatic and symptomatic disease group is mainly due to an increase of similar molecular and cellular functions rather than a difference in molecular and cellular functions, the exception being genes involved in RNA post-transcriptional regulation, protein degradation, and energy production that are significantly associated with symptomatic disease. Canonical pathway analysis with IPA showed that the IL-4 and chemokine signaling pathways are significantly associated with pre-symptomatic disease; while pyrimidine metabolism and the natural killer cell signaling pathway are significantly associated with symptomatic disease.

The IPA biomarker analysis tool also allowed for the identification of potential biomarkers for presymptomatic (Table 4) and symptomatic disease detection (Table 5).

It is indicated in Tables 4 and 5 whether the listed candidate biomarkers have been detected in various bodily fluids such as blood, bronchoalveolar lavage fluid, plasma, serum, or sputum. The genes are ranked based on the fold difference between disease and normal control group. Based on the functional analysis described herein, it is likely that during the course of disease, the expression levels of various sets of genes simply reach the necessary threshold to be statistically detected by these comparisons to normal controls, allowing for the development of early diagnosis markers for clinically asymptomatic patients.

These results demonstrate that the peripheral blood transcriptome distinguishes individuals with the familial form of IPF from non-diseased normal controls. Although pre-symptomatic and symptomatic disease were not clearly distinguished based on the expression profiles, these findings indicate that it may be possible to detect the disease before symptoms occur simply by analyzing the peripheral blood of an individual. The ability to use peripheral blood to detect FIP could have a substantial impact on the diagnosis, treatment, and management of this disease, and should be generalizable to other forms of IIP.

In this study, the differentially expressed genes in pre-symptomatic and symptomatic IPF are a valuable resource for selection of peripheral blood candidate biomarkers. Interestingly, MALAT1, a transcript up-regulated in pre-symptomatic disease, has been identified as a prognostic parameter for patient survival in stage I non-small cell lung carcinoma. The novel MALAT1 transcript is a non-coding RNA and MALAT1 transcripts are conserved across several species, implying an important function. This gene has not previously been implicated in IIP and emphasizes the potential role of non-coding RNAs in pulmonary fibrosis. Other genes up-regulated in pre-symptomatic and symptomatic disease are Annexin I (ANXA1) and beta catenin (CTNNB1). ANXA1 has been detected in bronchoaveolar lavage fluid of patients with ILD and belongs to a family of calcium (2+)-dependent phospholipid binding proteins acting as an inhibitor of phospholipase A2. The up-regulation of CTNNB1 in pulmonary fibrosis implicates the Wnt/catenin signaling pathway in disease pathogenesis. This pathway has been proposed for therapeutic intervention in IPF. Pathway analysis with IPA demonstrated that only a few pathways are well represented in the generated disease-stage specific gene lists. Together the IL-4, chemokine and natural killer cell signaling pathways indicate that the immune response plays a role in IPF pathogenesis and can be detected in peripheral blood transcriptional profiles of IPF patients.

The gene expression profiles have allowed for the identification of genes and pathways that are potentially important in the pathogenesis of FIP. Some of these genes might play an important role in disease development and some could be useful as disease biomarkers. Overall, these findings of an IPF peripheral blood molecular signature indicates that the development of a blood test for FIP, and even IPF, is feasible.

### Example 6

### Peripheral Blood Biomarkers Differentiate Extent of Disease for Idiopathic Pulmonary Fibrosis (IPF)

The majority of patients diagnosed with idiopathic pulmonary fibrosis (IPF) have a mortality rate of 3-5 years following diagnosis. Confirmatory diagnosis often requires invasive surgical lung biopsy which can cause complications, is costly, may result in delayed diagnosis and treatment, and has controversial accuracy. Peripheral blood biomarkers (PBB) have been identified and validated utilizing gene expression microarray profiling that distinguishes extent of disease in IPF. These validated peripheral blood biomarkers will translate into a widely available diagnostic blood test, transform the diagnostic approach to IPF by decreasing the time to diagnosis, diminish the need for invasive lung biopsies and provide the means to make a more accurate diagnosis.

Rationale.

Idiopathic pulmonary fibrosis (IPF) is a chronic disease of unknown etiology and is characterized by fibrosis or progressive scarring of the lung parenchyma, resulting in reduced gas diffusion and loss of lung volume. Ultimately, this fibrosis leads to respiratory failure resulting in an average mortality rate of 3.0 years following diagnosis. Currently, invasive lung biopsy is considered the gold standard and necessary in approximately half of the individuals. However invasive lung biopsy can cause complications, is not always accurate, is very costly, and often results in delayed diagnosis and treatment. Thus, the development and validation of peripheral blood biomarkers will allow molecular differentiation to distinguish between mild and severe forms of IPF.

Objective:

The objective of this study was to identify and validate molecular peripheral blood biomarkers utilizing microarray expression profiling that distinguishes extent of disease and disease progression in confirmed idiopathic pulmonary fibrosis patients.

Method:

Gene expression microarray profiles were generated utilizing peripheral blood RNA from 71 probable or definite clinically confirmed idiopathic pulmonary fibrosis patients. Expression profiles were correlated with percent predicted D_{L}CO and percent predicted FVC to identify biomarkers that differentiate extent of disease in the peripheral blood cohort and delineate disease progression. Differentially expressed transcripts of interest were validated via qRT-PCR.

Results.

Thirteen differentially expressed transcript identifiers were found between the mild and severe IPF cohort when categorized by D_{L}CO measurements differentiating extent of disease. Two differentially expressed transcripts, DEFA3 and FLJ11710, were found in common when comparisons were made between normal controls, mild IPF and severe cases of IPF to monitor IPF disease progression. Fold-change comparisons show an up-regulation in DEFA3 expression from normal controls through severe IPF disease, while FLJ11710 demonstrates a down regulation from normal controls through severe IPF cases.

Conclusion:

The peripheral blood transcriptome can distinguish extent of disease in individuals with IPF when samples were correlated with percent predicted D_{L}CO. The ability to use a peripheral blood biomarker to monitor disease progression for IPF could have a substantial impact on the diagnosis, treatment and management of this disease, and be generally applicable to other subtypes of idiopathic interstitial pneumonias.

Introduction.

Idiopathic Pulmonary Fibrosis (IPF) is categorized as an Interstitial Lung Disease (ILD) and is the most common subtype of Idiopathic Interstitial Pneumonias (IIP), encompassing nearly 71% of the total cases [1-5]. Prevalence estimates show that 20 per 100,000 males and 13 per 100,000 females have the disease [1]. IPF is a chronic disease of unknown etiology that is characterized by irreversible progressive fibrosis of the lung parenchyma and a disease that is unresponsive to therapeutic agents. The current hypothesis is fibroblastic foci are the active sites of disease progression which are caused by abnormal extracellular matrix remodeling [6, 7].

Of the IIPs, IPF has the least favorable prognosis with an average mortality rate of 3 years following diagnosis [8, 9]. Similar to those of other lung diseases, notable prognostic indicators of IPF include progressive deterioration of clinical symptoms such as dyspnea (shortness of breath) and pulmonary function [10, 11]. While dyspnea scoring has been used as a predictor of survival in IPF patients, its utilization as an unambiguous prognostic indicator is unrealistic as its metric is highly subjective and based on the individual's discernment of what constitutes shortness of breath [12]. Pulmonary function tests such as Diffusing Lung Capacity for Carbon Monoxide (D_{L}CO) and Forced Vital Capacity (FVC) have been utilized as predictive indicators [13, 14]. Studies demonstrate that a D_{L}CO of <35% or a decline in D_{L}CO>15% within a year period correlate with increased mortality, while a decline of >10% in FVC over a six month period was indicative of mortality [12, 15]. Randomized prospective controlled clinical trials in IPF have demonstrated significant differences in the rate of decline in lung function among the placebo arms of the trials, indicating there is substantial disease heterogeneity within IPF. Biomarkers that measure disease stage and activity would assist in understanding the effects of novel treatments, and the design of clinical trials with homogenous placebo and treatment groups.

In order to effectively make an early accurate diagnosis, monitor disease progression, and develop effective treatments for IPF, it is necessary to correlate underlying cellular, molecular, and genetic mechanisms via biomarker identification and monitoring to assess a biological state associated with IPF. Rosas and coworkers (2009) observed the differential expression of MMP7, MMP1, MMP8, IGFBP1, and TNFRSF1A proteins in the peripheral blood between familial interstitial pulmonary fibrosis patients and normal controls. However, the use of these biomarkers to differentiate disease severity or extent of disease within the IPF cohort was not addressed [9].

Therefore, it was hypothesized that peripheral blood biomarkers will identify disease stage (early or late), and allow monitoring for progression of disease. Such a biomarker of idiopathic pulmonary fibrosis would allow for earlier diagnosis at a more readily treatable stage of their disease, or identify those at risk for rapid disease progression.

Study Populations.

Seventy-one peripheral blood RNA specimens were collected from individuals enrolled in either the Interstitial Lung Disease (ILD) or the Familial Pulmonary Fibrosis (FPF) Programs conducted at National Jewish Health, Duke University and Vanderbilt University. All blood collections were approved by the respective Institutional Review Board (IRB) and all subjects provided informed consent. Only one specimen per family was utilized from the FPF repository to comprise the respective cohorts. Individual samples had a consensus diagnosis of probable or definite IPF that was confirmed by high resolution computed tomography (HRCT) scans and/or lung biopsy. Clinical and demographic information for the peripheral blood specimens and normal controls are provided in Table 6. Specimens were further categorized based on percent predicted D_{L}CO and FVC as shown in Tables 7 and 8. The microarrays were utilized to generate peripheral blood gene expression profiles on individuals with percent predicted D_{L}CO≧65% (N=16) or FVC≧75% (N=27) and D_{L}CO≦35% (N=15), FVC≦50% (N=13). All of the IPF profiles were also compared to age and gender matched non-diseased, healthy controls (N=31).

### Expression Profiling.

Peripheral Blood RNA Isolation and Purification.

Peripheral blood samples were collected in PAXgene RNA tubes (PreAnalytiX, 762165) and stored at −80° C. until needed. PAXgene RNA tubes were thawed at room temperature for a minimum of two hours prior to RNA extraction and purification. RNA extraction and purification was performed manually utilizing the PAXgene Blood RNA kit (PreAnalytiX, 762164). Specifically, the peripheral blood samples were centrifuged (3000×g) for 10 minutes to pellet cells and the supernatant discarded. Four mL of RNAse free water was added to the pellet and dissolved by vortexing. The mixture was centrifuged again for an additional 10 minutes (3,000×g) and supernatant discarded. The pellet was re-suspended in 350 μL of BR1 re-suspension buffer and vortexed until the pellet dissolved. The mixture was transferred to a 1.5 mL microcentrifuge tube, and 300 μL of BR2 buffer and 40 μL of proteinase K were added. The mixture was vortexed and incubated at 55° C. for 10 minutes. The mixture was transferred to a Paxgene Shredder spin column and centrifuged for 3 minutes (13,000 rpm). Without disrupting the pellet, the resulting supernatant of the flow through was transferred to a clean 1.5 mL microcentrifuge tube and 350 μL of 96% ethanol added. Seven hundred μL of the mixture was transferred to a Paxgene RNA spin column and centrifuged for 1 minute (13,000 rpm). After centrifugation, the RNA spin column was placed in a clean processing tube and the remainder of the mixture was centrifuged for 1 minute (13,000 rpm). The RNA spin column was placed in a clean processing tube, 350 μL of BR3 buffer added and centrifuged for 1 minute (13,000 rpm). A mixture consisting of 70 μL of RDD buffer and 10 μL of DNAse I was added to the RNA spin column and incubated for 15 minutes at room temperature. The RNA spin column was transferred to a clean processing tube, 350 μL of BR3 buffer added and centrifuged for 1 minute (13,000 rpm). After replacement with a clean processing tube, 500 μL of BR4 buffer was added to the RNA spin column and centrifuged for 1 minute (13,000 rpm). The RNA spin column was transferred to a clean processing tube, an additional 500 μL of BR4 buffer added and centrifuged for 3 minutes (13,000 rpm). The RNA spin column was transferred to a clean processing tube and centrifuged for 1 minute. The RNA spin column was transferred to a 1.5 mL microcentrifuge tube, 40 μL of BR5 buffer added and centrifuged for 1 minute (13,000 rpm). This step was repeated twice into the same 1.5 mL microcentrifuge tube. The resulting 80 μL of eluate was incubated at 65° C. for 5 minutes and immediately put on ice for total RNA quantification and quality characterization.

Total RNA Quantification and Quality Characterization.

Quantification of total RNA was measured via the Nanodrop ND-1000 spectrophotometer (NanoDrop Technologies, Wilmington, Del.). Quality of the RNA was assessed with a RNA 6000 NanoChip (Agilent, Palo Alto, Calif.) on the 2100 Bioanalyzer (Agilent, Palo Alto, Calif.) by ratio comparison of the 18S and 28S rRNA bands.

Microarrays. Agilent Whole Human Genome Oligonucleotide Microarrays (G4112F Agilent, Palo Alto, Calif.), containing 4×44K 60-mer oligonucleotides representing over 44,000 human genes and transcripts, were used to determine gene expression levels in peripheral blood. Twenty-five to 200 ng of total RNA was used as a template for synthesis of cDNA and amplified utilizing the One Color Low Input Agilent Quick Amp Labeling Kit (5190-2305). The cDNA was used as a template to generate Cy3-labeled cRNA for hybridization. The Agilent One Color RNA Spike-In Kit (5188-5282), which consisted of a set of 10 positive control transcripts (polyadenylated transcripts derived from the Adenovirus E1A gene), was utilized to provide positive controls for monitoring the one color gene expression microarray workflow from sample amplification and labeling to microarray processing. The Agilent one-color microarray based gene expression analysis used the thermocycler protocol and was followed per manufacturer's instructions. For each sample, 1.65 μg of Cy3 labeled cRNA was fragmented and hybridized for 17 hours in a rotating hybridization oven. Slides were washed and then scanned with an Agilent Scanner. Data and quality control metrics for the microarrays were generated using the Agilent Feature Extraction software (10.7.1.1), using the 1-color defaults for all parameters.

Normalization. Microarray quantile normalization with quality controls was performed in the R statistical environment (http://www.r-project.org) using the Agi4x44PreProcess package downloaded from the Bioconductor web site (http://bioconductor.orgi). Normalization and further filtering steps were based on those described in the Agi4x44PreProcess reference manual.

Microarray Data Analysis.

Analysis was performed on the microarray data sets utilizing the Multi-Experiment Viewer (MeV) software package [16]. Significant analysis of microarrays (SAM) with a false discovery rate (FDR) of 5% was utilized within the program to identify genes that were differentially expressed between IPF samples as categorized based on percent predicted D_{L}CO and FVC stated previously. All IPF samples were also compared to normal controls to identify differentially expressed genes. Principle component analysis (PCA) was performed on all SAM analyses to identify outliers.

Gene Ontology and Functional Network Analysis.

Data were analyzed through the use of Ingenuity Pathways Analysis (Ingenuity Systems, www.ingenuity.com). Ingenuity Pathway Analysis (IPA) is a web-based application that enables the visualization, discovery and analysis of molecular interaction networks within gene expression profiles. All generated gene lists and corresponding expression levels, represented as the log₂ ratios, were uploaded within the IPA database for further analysis. Both gene symbols and gene bank accession numbers were used with no apparent differences in results. These genes, called focus genes, were overlaid onto a global molecular network developed from information contained in the Ingenuity knowledge base. The IPA knowledge base represents a proprietary ontology of over 600,000 classes of biologic objects spanning genes, proteins, cells and cell components, anatomy, molecular and cellular processes, and small molecules. Networks of the focus genes were then algorithmically generated based on their connectivity. The Functional Analysis of a network identified the biological functions and/or diseases that were most significant to the genes in the network. The network genes associated with biological functions and/or diseases in the Ingenuity knowledge base were considered for the analysis. Fischer's exact test was used to calculate a P-value determining the probability that each biological function and/or disease assigned to that network is due to chance alone. Canonical Pathways Analysis identified the pathways from the Ingenuity Pathways Analysis library of canonical pathways that were most significant to the dataset. The significance of the association between the dataset and the canonical pathway was measured in two ways. 1) a ratio of the number of genes from the dataset that map to the pathway divided by the total number of molecules that exist in the canonical pathway is displayed. 2) Fischer's exact test was used to calculate a P-value. Biomarker Analysis allows the identification of the most relevant molecular biomarker candidates from a dataset based on contextual information such as mechanistic association with a disease or detection in bodily fluids.

Validation.

Quantitative real-time PCR was utilized to confirm differential expression of genes found by microarray analysis. Total RNA extracted from peripheral blood was reverse transcribed to cDNA using the High Capacity Reverse Transcription kit (Applied Biosystems, Foster City, Calif.) using standard protocols. Quantitative real-time PCR using SYBR Green fluorescent dye was performed on an ABI 7900HT Fast Real-Time PCR Detection System (Applied Biosystems, Foster City, Calif.) with forty cycles of amplification and data acquisition. Each 20 μL reaction contained 1×SYBR Green PCR Master Mix (Applied Biosystems, Foster City, Calif.), 10 ng cDNA, and 0.5 μM each forward and reverse primer (Integrated DNA Technologies, Coralville, Iowa). Primer design was optimized with Primer-Blast software (http://www.ncbi.nlm.nih.gov/tools/primer-blast/) to span exon-exon junctions where possible. All assays were performed in duplicate and data were analyzed by the ΔΔCt method utilizing glyceraldehyde 3 phosphate dehydrogenase (GAPDH) as an endogenous control.

Extent of Disease Analysis Comparison.

First an investigation was done to determine whether peripheral blood gene expression profiles could be utilized to differentiate extent of disease when IPF samples were categorized by pulmonary function measurements. Peripheral blood gene expression profiles were compared for mild and severe cases of IPF based on percent predicted FVC and percent predicted D_{L}CO.

Significant analysis of microarrays revealed no differentially expressed transcripts with less than a 5% false discovery rate between peripheral blood samples when IPF patients were categorized by percent predicted FVC (N=27 and N=13). However, significant analysis of microarrays of IPF samples, when categorized by percent predicted D_{L}CO (mild IPF N=16 and severe IPF N=15), demonstrated a total of 13 differential expressed transcripts with less than a 5% false discovery rate. Table 9 lists all differentially expressed genes found between mild and severe cases of IPF. Principle component analysis was performed to determine outliers in the data set based on severity of disease categorization. Results demonstrate that one IPF case appears to be clinically misclassified as a mild case of IPF.

Hierarchal clustering was performed simultaneously on both the differentially expressed genes and associated disease severity categorization to determine disease-specific patterns that correlate to IPF disease diagnosis. Results from this statistical approach organized patients into six major groups. The significance in this analysis is that it demonstrates disease categorization based on percent predicted D_{L}CO alone is insufficient to categorize extent of disease. This is evident by three mild cases of IPF having greater similarity to more severe cases of IPF when molecular differentiation is considered in the analysis.

This list of differentially expressed genes was subjected to a functional analysis. The functional analysis tool of the Ingenuity Pathway Analysis (IPA) software was utilized to identify common associates, biological functions and diseases to the experimental results. The functional analysis tool also calculates a significance value that is a measure for the likelihood that the association between a set of genes and a given process is due to random chance. Results show that of the 13 differentially expressed transcript identifiers found between the mild and severe IPF cohort, 10 had annotations representing a gene, protein or chemical that was able to be mapped to an associated network. The associated network functions included 1) inflammatory response, cellular movement and immune trafficking; 2) genetic disorder, inflammatory and respiratory diseases; and 3) cell-to-cell signaling, tissue development and cellular movement. Table 10 lists the associated p-value range with the corresponding top bio-functions in the networks.

Of particular IPF interest is the up-regulation of genes between the IPF cohort (D_{L}CO≧65% and D_{L}CO≦35%) which code for the carcinoembryonic cell adhesion molecule 6 (CEACAM6, a.k.a. CD66C, CEAL and NCA) to differentiate extent of disease. Investigation shows that CEACAM6 is not found to be differentially expressed between normal controls compared to samples in the IPF cohort which have a D_{L}CO≧65%. This gene encodes glycosylated, glycosylphosphatidylinositol (GPI) anchored proteins that have been found to be expressed in alveolar epithelial cells [21-23].

Differential expression analysis demonstrated the up-regulation of the cathelicidin antimicrobial peptide (CAMP, a.k.a. CAP18, CAP-18/LL-37, CATHELICIDIN, CRAMP, FALL-39, hCAP-18 and HSD26) between the IPF cohort and when the IPF cohort had a D_{L}CO≦35% when compared to normal controls. This gene has been utilized as a biomarker in serum for lung cancer [24] and has also been reported to be up-regulated in cystic fibrosis [25] and severe acute respiratory syndrome [26]. While the CAMP gene shows no differential expression in the mild IPF cohort when the D_{L}CO is ≧65% compared to normal controls, it has been found to be expressed in lung tissue, peripheral blood, plasma as well as bronchoalveolar lavage fluid (BAL).

Disease Progression Analysis.

Next it was investigated whether there were differentially expressed transcripts in the peripheral blood which could be utilized as potential biomarkers to monitor disease progression.

Significant analysis of microarrays of the mild IPF cohort, when categorized by percent predicted D_{L}CO (mild TPF N=16) compared to normal controls (N=31) produced a total of 4,809 differential expressed transcripts with less than a 5% false discovery rate. SAM was also performed on the severe IPF cases, when categorized by percent predicted D_{L}CO (severe IPF N=15) compared to normal controls (N=31). Results indicated a total of 5,330 differentially expressed transcripts with the same FDR cutoff. Tables 12 and 13 show differentially expressed transcripts with at least a 2-fold difference for the mild and severe IPF cases compared to normal controls, respectively.

The general comparison tool of the IPA software was utilized to identify the intersection or common differentially expressed transcripts between the three gene lists. Table 11 provides the log₂ ratio fold-changes between the three comparisons for all potential disease progression biomarkers identified. Results show that only two differentially expressed transcripts, DEFA3 and FLJ11710, were common to all three lists. Fold-change comparisons demonstrate an up-regulation in DEFA3 expression from normal controls through severe IPF disease, while FLJ11710 demonstrates a down regulation from normal controls through severe IPF cases.

While FLJ11710 demonstrates a down regulation from normal controls through severe cases of IPF, little is known about its molecular functionality. It is reported to have protein-protein interactions with a disintegrin and metalloproteinase (ADAM 15), alcohol group acceptor phosphotransferase (PAK2) and nuclear transport factor 2 (NUTF2), all of which have involvement in cell-to-cell signaling, tissue development and cellular movement [27].

However, human neutrophil α-defensins (also designated HNPs) are small, cationic, cysteine-rich antimicrobial peptides that play important roles in innate immunity against infectious microbes such as bacteria, fungi and enveloped viruses [28]. In humans, a-defensins 1-4 are primarily found in neutrophils and in the epithelia of mucosal surfaces such as those found in the respiratory tract [29, 30]. These α-defensins are synthesized as inactive precursors consisting of 29-42 amino acid residues and are activated by proteolytic cleavage via MMP7 [31]. FIG. 5 shows the pathway interaction of MMP7 with the alpha defensins. Interestingly, it has been previously observed that α-defensin levels in bronchial alveolar lavage and/or plasma are increased in fibrotic lung diseases like idiopathic pulmonary fibrosis (IPF) and that a significant amount of α-defensins can be found outside neutrophils in fibrotic foci in the lungs of patients with IPF [32]. In addition, it has been reported that inflammatory lung diseases with neutrophil infiltration are complicated with fibroproliferative foci and α-defensins may contribute an important role in their formation [33, 34].

Conclusions.

Results provided herein demonstrate that the peripheral blood transcriptome can distinguish extent of disease in individuals with IPF when samples were correlated with percent predicted D_{L}CO. The ability to use a peripheral blood biomarker to monitor disease progression for IPF could have a substantial impact on the diagnosis, treatment, staging and management of this disease, and perhaps be generally applicable to other subtypes of idiopathic interstitial pneumonias.

Any patents, patent publications or non-patent publications mentioned in this specification are indicative of the level of those skilled in the art to which the invention pertains. These patents and publications are herein incorporated by reference to the same extent as if each individual patent or publication was specifically and individually indicated to be incorporated by reference.

One skilled in the art will readily appreciate that the present invention is well adapted to carry out the objects and obtain the ends and advantages mentioned, as well as those inherent therein. The present examples along with the methods, procedures, treatments, molecules, and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

### REFERENCES

1. Anonymous, American Thoracic Society/European Respiratory Society International Multidisciplinary Consensus Classification of the Idiopathic interstitial Pneumonias This Joint Statement of the American Thoracic Society (ATS), and the European Respiratory Society (ERS) was adopted by the ATS Board of Directors, June 2001 and by The ERS Executive Committee, June 2001. Am. J. Respir. Crit. Care Med., 2002. 165(2): p. 277-304.
2. Bjorker, J. A., et al., Prognostic Significance of Histopathologic Subsets in Idiopathic Pulmonary Fibrosis. Am. J. Respir. Crit. Care Med., 1998. 157(1): p. 199-203.
3. Flaherty, K. R., et al., Histopathologic Variability in Usual and Nonspecific Interstitial Pneumonias. Am. J. Respir. Crit. Care Med., 2001. 164(9): p. 1722-1727.
4. Nicholson, A. G., et al., Nonspecific Interstitial Pneumonia-Nobody Said It's Perfect. Am. J. Respir. Crit. Care Med., 2001. 164(9): p. 1553-1554.
5. Nicholson, A. G., et al., The Relationship between Individual Histologic Features and Disease Progression in Idiopathic Pulmonary Fibrosis. Am. J. Respir. Crit. Care Med., 2002. 166(2): p. 173-177.
6. Moeller, A., et al., Circulating Fibrocytes are an Indictator of Poor Prognosis in Idiopathic Pulmonary Fibrosis. Am J Respir Crit. Care, 2009. 179: p. 588-594.
7. King, T. E., Jr., et al., Idiopathic Pulmonary Fibrosis Relationship between Histopathologic Features and Mortality. Am. J. Respir. Crit. Care Med., 2001. 164(6): p. 1025-1032.
8. Boon, K., et al., Molecular Phenotypes Distinguish Patients with Relatively Stable from Progressive Idiopathic Pulmonary Fibrosis (IPF). PLoS One, 2009. 4(4): p. e5134.
9. Rosas, I. O., et al., MMP1 and MMP7 as Potential Peripheral Blood Biomarkers in Idiopathic Pulmonary Fibrosis. PLoS Medicine, 2008. 5(4): p. 623-633.
10. Gross, T. J. and G. W. Hunninghake, Idiopathic Pulmonary Fibrosis. N. Engl. J. Med., 2001. 345(7): p. 517-525.
11. Tzilas, V., et al., Prognostic Factors in Idiopathic Pulmonary Fibrosis. The American Journal of the Medical Sciences, 2009. 338 (6): p. 481-485
12. Collard, H. R., et al., Changes in Clinical and Physiologic Variables Predict Survival in Idiopathic Pulmonary Fibrosis. Am. J. Respir. Crit. Care Med., 2003. 168(5): p. 538-542.
13. Jegal, Y., et al., Physiology Is a Stronger Predictor of Survival than Pathology in Fibrotic Interstitial Pneumonia. Am. J. Respir. Crit. Care Med., 2005. 171(6): p. 639-644.
14. Latsi, P. I., et al., Fibrotic Idiopathic Interstitial Pneumonia: The Prognostic Value of Longitudinal Functional Trends. Am. J. Respir. Crit. Care Med., 2003. 168(5): p. 531-537.
15. Flaherty, K. R., et al., Idiopathic Pulmonary Fibrosis: Prognostic Value of Changes in Physiology and Six-Minute-Walk Test. Am. J. Respir. Crit. Care Med., 2006. 174(7): p. 803-809.
16. Saeed, A., et al., TM4: a free, open-source system for microarray data management and analysis. Biotechniques, 2003. 34(2): p. 374-378.
17. Munson, J. C., Combined pulmonary fibrosis and emphysema: a high-pressure situation. European Respiratory Journal, 2010. 35(1): p. 9-11.
18. Cottin, V., et al., Combined pulmonary fibrosis and emphysema: a distinct underrecognised entity. Eur. Respir. J., 2005. 26(586-593).
19. Doherty, M. J., et al., Cryptogenic fibrosing alveolitis with preserved lung volumes. Thorax, 1997. 52(11): p. 998-1002.
20. Mejia, M., et al., Idiopathic Pulmonary Fibrosis and Emphysema: Decreased Survival Associated With Severe Pulmonary Arterial Hypertension. Chest, 2009. 136(1): p. 10-15.
21. Kammerer, R., et al., Identification of allelic variants of the bovine immune regulatory molecule CEACAM1 implies a pathogen-driven evolution. Gene 2004. 339: p. 99-109.
22. Kuroki, M., et al., Identcation and comparison of residues critical for cell-adhesion activities of two neutrophil CD66 antigens, CEACAM6 and CEACAM8. J Leukoc Biol 2001. 70: p. 543-550.
23. Venkatadri, K., et al., Ballard Carcinoembryonic cell adhesion molecule 6 in human lung: regulated expression of a multifunctional type II cell protein Physiol Lung Cell Mol Physiol, 2009. 296 (6): p. L1019-L1030.
24. Coffelt, S. B. and A. B. Scandurro, Tumors sound the alarmin(s). Cancer Res 2008. 68(16): p. 6482-6485.
25. Felgentreff, K., et al., The antimicrobial peptide cathelicidin interacts with airway mucus. Peptides 2006. 27(12): p. 3100-3106.
26. Reghunathan, R., et al., Expression profile of immune response genes in patients with Severe Acute Respiratory Syndrome. BMC Immunol, 2005. 6(2).
27. Stelzl, U., et al., A human protein-protein interaction network: a resource for annotating the proteome. Cell, 2005. 122(6): p. 957-968.
28. Bevins, C. L., Paneth cell defensins: key effector molecules of innate immunity. Antimicrobial Peptides, 2006. 34(2): p. 253-266.
29. Amenomori, M., et al., Differential effects of human neutrophil peptide-1 on growth factor and interleukin-8 production by human lung fibroblasts and epithelial cells. Experimental Lung Research, 2010. 36(7): p. 411-419.
30. Zitvogel, L., O. Kepp, and G. Kroemer, Decoding cell death signals in inflammation and immunity. Cell, 2010. 140(6): p. 798-804.
31. Wilson, C. L., et al., Differential Processing of α-and β-Defensin Precursors by Matrix Metalloproteinase-7 (MMP-7). Biol. Chem., 2009. 284(13): p. 8301-8311.
32. Mukae, H., et al., Raised plasma concentrations of alpha-defensins in patients with idiopathic pulmonary fibrosis. Thorax 2002. 57: p. 623-628.
33. Han, W., et al., α-Defensins increase lung fibroblast proliferation and collagen synthesis via the β-catenin signaling pathway. FEBS J, 2009, 276(22): p. 6603-6614.
34. Van Wetering, S., G. S. Tjabring a, and P. S. Hiemstra, Interactions between neutrophil-derived antimicrobial peptides and airway epithelial cells. J. Leukoc. Biol., 2005. 77: p. 444-450.

TABLE 1
Clinical and demographic variables.

| | Pre- | | |
|---|---|---|---|
| Variable | symptomatic | Symptomatic | Control |
| Age | 63.7 ± 8.7 | 59.9 ± 8.2 | 59.4 ± 11 |
| Sex male/female | 3/4 | 3/4 | 6/5 |
| Smoking status | | | |
| never | 2 | 5 | 5 |
| ever | 4 | 2 | 5 |
| current | 1 | 0 | 1 |
| Dyspnea rating | 0-1 | 4-5 | nd |
| % predicted | 79.3 ± 12.4 | 39.4 ± 10.8 | nd |
| DLCO | | | |
| Diagnosis | FIP-IPF | FIP-IPF | normal |

Definitions of abbreviations:
PF = pulmonary fibrosis;
nd = no data available.

TABLE 2
Candidate Markers for pre-symptomatic disease when compared to healthy controls.

| Gene | | | Fold | | | | |
|---|---|---|---|---|---|---|---|
| Symbol | Gene Description | Location | Change | B | BAL | P/S | Sp |
| PAEP | progestagen-associated endometrial protein | Extracellular Space | 2.492 | x | | x | |
| HOOK3 | hook homolog 3 (Drosophila) | Cytoplasm | 2.078 | x | | | |
| FAM13A1 | family with sequence similarity 13, member A1 | Unknown | 1.998 | x | | x | |
| RHCG | Rh family, C glycoprotein | Plasma Membrane | 1.903 | x | | | |
| HLA-DRA | major histocompatibility complex, class II, DR alpha | Plasma Membrane | 1.871 | x | x | | |
| IREB2 | iron-responsive element binding protein 2 | Cytoplasm | 1.864 | x | | x | |
| CLINT1 | clathrin interactor 1 | Cytoplasm | 1.845 | x | | x | |
| CRIP1 | cysteine-rich protein 1 (intestinal) | Cytoplasm | 1.773 | | x | | |
| PRKCI | protein kinase C, iota | Cytoplasm | 1.756 | x | | x | |
| BDP1 | B double prime 1, subunit of RNA, po [symbol] III transcription Factor IIIB | Nucleus | 1.705 | x | x | x | |
| MAN2A1 | mannosidase, alpha, class 2A, member 1 | Cytoplasm | 1.678 | x | | | |
| SLC16A6 | solute carrier family 16, member 6 | Plasma Membrane | 1.657 | x | | x | |
| TNFAIP3 | tumor necrosis factor, alpha-induced protein 3 | Nucleus | 1.644 | x | | | |
| HLA-DOA | major histocompatibility complex, class II, DO alpha | Plasma Membrane | 1.643 | x | | | |
| MLL | myeloid/lymphoid or mixed-lineage leukemia ( [symbol] homolog) | Nucleus | 1.625 | x | | x | |
| CYSLTR1 | cysteinyl leukotriene receptor 1 | Plasma Membrane | 1.615 | x | | | |
| MEF2C | myocyte enhancer factor 2C | Nucleus | 1.606 | x | | | |
| UBE3A | ubiquitin protein ligase E3A (Angelman syndrome) | Nucleus | 1.605 | x | | x | |
| DZIP3 | DAZ interacting protein 3, zinc finger | Cytoplasm | 1.593 | x | | x | |
| RPL10L | ribosomal protein L10-like | Nucleus | 1.589 | x | | x | |
| ITPR2 | inositol 1,4,5-triphosphate receptor, type 2 | Cytoplasm | 1.584 | x | | x | |
| PSMA2 | proteasome (prosome, macropain) subunit, alpha type, 2 | Cytoplasm | 1.563 | | x | | |
| PEA15 | phosphoprotein enriched in astrocytes 15 | Cytoplasm | 1.552 | | x | | |
| PPIA | peptidylprolyl isomerase A (cy [symbol] ph [symbol] n A) | Cytoplasm | 1.546 | x | x | | x |
| YME1L1 | YME1-like 1 (S. cerevisiae) | Cytoplasm | 1.536 | x | | x | |
| NKTR | natural killer-tumor recognition sequence | Plasma Membrane | 1.522 | x | | x | |
| M6PR | mannose-6-phosphate receptor (cation dependent) | Cytoplasm | 1.513 | x | | | |
| ROD1 | ROD1 regulator of differentiation 1 (S. pombe) | Nucleus | 1.508 | x | | | |
| ADAMTS7 | ADAM metallopeptidase with thrombospondin type 1 motif, 7 | Extracellular Space | −1.496 | x | | x | |
| RAB11B | RAB11B, member RAS oncogene family | Cytoplasm | −1.564 | x | | | |
| PPP1CB | protein phosphatase 1, catalytic subunit, beta isoform | Cytoplasm | −1.597 | x | | x | |
| FBXO38 | F-box protein 38 | Nucleus | −1.627 | x | | x | |
| HLA-G | major histocompatibility complex, class I, G | Plasma Membrane | −1.802 | x | | | |
| PNPLA2 | pata [symbol] -like phospholipase domain containing 2 | Cytoplasm | −1.802 | x | | x | |
| SLC39A7 | solute carrier family 39 (zinc transporter), member 7 | Plasma Membrane | −1.803 | x | | x | |
| FN1 | fibronectin 1 | Plasma Membrane | −2.616 | x | x | x | |

Based on the interature available in the IPA database the cellular localization and detection in bodily fluids is indicated.
Fold change is represented as the difference in expression level when compared to normal
B = blood;
BAL = Branchoalveolar Lavage Fluid;
P/S = Plasma/Serum;
SP = Sputum. [symbol]
indicates data missing or illegible when filed

TABLE 3
Unique Candidate Biomarkers for late disease

| Gene | | | Fold | | | | |
|---|---|---|---|---|---|---|---|
| Symbol | Gene Description | Location | Change | B | BAL | P/S | Sp |
| PLAT | plasminogen activator, tissue | Extracellular Space | 11.377 | x | | x | |
| SIGLEC12 | sialic acid binding Ig-like lectin 12 | Plasma Membrane | 3.707 | x | | | |
| PTGFR | prostaglandin F receptor (FP) | Plasma Membrane | 2.668 | x | | | |
| TFEC | transcription factor EC | Nucleus | 2.584 | x | | | |
| ITGA1 | integrin, alpha 1 | Plasma Membrane | 2.579 | x | | x | |
| HNMT | histamine N-methyltransferase | Cytoplasm | 2.494 | | x | | |
| CLEC4G | C-type lectin superfamily 4, member G | Plasma Membrane | 2.43 | x | | | |
| LY96 | lymphocyte antigen 96 | Plasma Membrane | 2.386 | x | | | |
| SMARCA2 | SWI/SNF related, matrix associated, a2 | Nucleus | 2.239 | x | | x | |
| MYL6B | myosin, light chain 6B non-muscle | Cytoplasm | 2.057 | x | | | |
| RHOU | ras homolog gene family, member U | Cytoplasm | 2.015 | x | | | |
| COTL1 | coactosin-like 1 (Dictyostelium) | Cytoplasm | 2.008 | x | x | | |
| GLRX | glutaredoxin (thioltransferase) | Cytoplasm | 1.962 | x | x | | |
| P4HA1 | procollagen-proline, 4-dioxygenase a1 | Cytoplasm | 1.946 | x | | x | |
| HEBP2 | heme binding protein 2 | Cytoplasm | 1.923 | | x | | |
| FCER1G | Fc fragment of IgE, high affinity | Plasma Membrane | 1.910 | x | x | | |
| NUDT2 | nudix-type motif 2 | Plasma Membrane | 1.901 | | x | | |
| SNX5 | sorting nexin 5 | Cytoplasm | 1.897 | x | | x | |
| NAIP | NLR family, apoptosis inhibitory protein | Cytoplasm | 1.89 | x | | x | |
| TRIM7 | tripartite motif-containing 7 | Cytoplasm | 1.883 | x | | x | |
| GAS8 | growth arrest-specific 8 | Cytoplasm | 1.842 | x | | x | |
| GTF2B | general transcription factor IIB | Nucleus | 1.776 | x | | x | |
| S100A8 | S100 calcium binding protein A8 | Cytoplasm | 1.715 | x | x | x | x |
| PGK1 | phosphoglycerate kinase 1 | Cytoplasm | 1.671 | x | x | x | x |
| SMARCD3 | SWI/SNF related, matrix associated d3 | Nucleus | 1.670 | x | | | |
| RIPK2 | receptor-interacting serine-threonine kinase 2 | Plasma Membrane | 1.654 | x | | | |
| NPM1 | nucleophosmin B23, numatrin | Nucleus | 1.646 | x | | | |
| CASP1 | caspase 1 (interleukin 1, beta, convertase) | Cytoplasm | 1.630 | x | | | |
| WWOX | WW domain containing oxidoreductase | Cytoplasm | 1.625 | x | | x | |
| TNFRSF10B | tumor necrosis factor receptor 10B | Plasma Membrane | 1.577 | x | | | |
| NPEPPS | aminopeptidase puromycin sensitive | Cytoplasm | 1.574 | | x | | |
| AIF1 | a [symbol] lograft inflammatory factor 1 | Nucleus | 1.566 | | x | | |
| AP3S1 | adaptor-related protein complex 3, sigma 1 | Cytoplasm | 1.524 | x | | | |
| CYP2D6 | cytochrome P450, family 2, subfamily D, 6 | Cytoplasm | 1.511 | x | | | |
| CRIPT | cysteine-rich PDZ-binding protein | Cytoplasm | 1.510 | x | | x | |
| CTRL | chymotrypsin-like | Extracellular Space | 1.504 | x | | | |
| BAIAP2 | BAI1-associated protein 2 | Plasma Membrane | 1.46 | | x | | |
| HK3 | hexokinase 3 (white cell) | Cytoplasm | 1.445 | x | x | x | |
| ZC3H12A | zinc finger CCCH-type containing 12A | Unknown | 1.400 | x | | x | |
| USP35 | ubiquitin specific peptidase 35 | Unknown | 1.395 | x | | x | |
| ZFP106 | zinc finger protein 106 homolog (mouse) | Cytoplasm | 1.351 | x | | | |
| NUDT3 | Nud [symbol] -type motif 3 | Cytoplasm | 1.299 | | x | | |
| DNM2 | dynamin 2 | Plasma Membrane | 1.288 | x | | | |
| SRF | serum response factor | Nucleus | −1.339 | x | | | |
| PRMT2 | protein arginine methyltransferase 2 | Nucleus | −1.353 | x | | x | |
| SSR1 | signal sequence receptor, alpha | Cytoplasm | −1.424 | x | | | |
| TCP1 | t-complex 1 | Cytoplasm | −1.452 | | x | | |
| APEH | N-acylaminoacyl-peptide hydrolase | Cytoplasm | −1.486 | | x | | |
| RPA1 | replication protein A1, 70 kDa | Nucleus | −1.491 | x | | | |
| SRPR | signal recognition particle receptor | Cytoplasm | −1.524 | x | | x | |
| HCGA1 | heterogeneous nuclear ribonucleoprotein A1 | Unknown | −1.765 | x | | | |
| SFPQ | splicing factor proline/glutamine-rich | Nucleus | −1.776 | x | | | |
| VEGFB | vascular endothelial growth factor B | Extracellular Space | −1.801 | x | x | x | |
| KIR3DL1 | ki [symbol] er cell immunoglobulin-like receptor, L1 | Plasma Membrane | −1.841 | x | | | |
| UCP2 | uncoupling protein | Cytoplasm | −1.888 | x | | | |
| KIR2DL2 | ki [symbol] er cell immunoglobulin-like receptor, L2 | Plasma Membrane | −2.125 | x | | | |
| INCENP | inner centromere protein antigens 135/155 kDa | Nucleus | −2.307 | x | | x | |
| KIR2DS2 | ki [symbol] er cell immunoglobulin-like receptor, S 2 | Plasma Membrane | −2.670 | x | | | |
| KIR2DS4 | ki [symbol] er cell immunoglobulin-like receptor, S4 | Plasma Membrane | −2.784 | x | | | |
| KIR3DL2 | ki [symbol] er cell immunoglobulin-like receptor, L2 | Plasma Membrane | −2.850 | x | | | |
| KIR2DS1 | ki [symbol] er cell immunoglobulin-like receptor, S1 | Plasma Membrane | −2.884 | x | | | |
| MC2R | melanocortin 2 receptor (adrenocorticotropic) | Plasma Membrane | −3.533 | x | | | |
| RAB3B | RAB3B, member RAS oncogene family | Cytoplasm | −6.996 | x | | | |

[symbol]

indicates data missing or illegible when filed

TABLE 4
Unique Candidate Biomarkers for pre-symptomatic disease when compared to healthy controls

| Gene | | | Fold | | | | |
|---|---|---|---|---|---|---|---|
| Symbol | Gene Description | Location | Change | B | BAL | P/S | Sp |
| PAEP | progestagen-associated endometrial protein | Extracellular Space | 2.492 | x | | x | |
| HOOK3 | hook homolog 3 (Drosophila) | Cytoplasm | 2.078 | x | | | |
| FAM13A1 | family with sequence similarity 13, member A1 | Unknown | 1.9 [symbol] | x | | x | |
| RHCG | Rh family, C glycoprotein | Plasma Membrane | 1.903 | x | | | |
| HLA-DRA | major histocompatibility complex, class II, DR alpha | Plasma Membrane | 1.871 | x | x | | |
| IREB2 | iron-responsive element binding protein 2 | Cytoplasm | 1.8 [symbol] 4 | x | | x | |
| CLINT1 | clathrin interactor 1 | Cytoplasm | 1.845 | x | | x | |
| CRIP1 | cy [symbol] -rich protein 1 (intestinal) | Cytoplasm | 1.773 | | x | | |
| PRKCI | protein kinase C, iota | Cytoplasm | 1.75 [symbol] | x | | x | |
| BDP1 | B double prime 1, subunit of transcription Factor IIIB | Nucleus | 1.705 | x | x | x | |
| MAN2A1 | mannosidase, alpha, class 2A, member 1 | Cytoplasm | 1. [symbol] 78 | x | | | |
| SLC16A6 | solute carrier family 16, member 6 | Plasma Membrane | 1.657 | x | | x | |
| TNFAIP3 | tumor necrosis factor, alpha-induced protein 3 | Nucleus | 1.644 | x | | | |
| HLA-DOA | major histocompatibility complex, class II, DO alpha | Plasma Membrane | 1.643 | x | | | |
| MLL | my [symbol] or [symbol] -lineage leukemia | Nucleus | 1.625 | x | | x | |
| CYSLTR1 | cysteinyl leuk [symbol] receptor 1 | Plasma Membrane | 1.615 | x | | | |
| MEF2C | myocyte enhancer factor 2C | Nucleus | 1.606 | x | | | |
| UBE3A | ubiquitin protein ligase E3A (Angelman syndrome) | Nucleus | 1.605 | x | | x | |
| DZIP3 | DAZ interacting protein 3, zinc finger | Cytoplasm | 1.593 | x | | x | |
| RPL10L | ribosomal protein L10-like | Nucleus | 1.589 | x | | x | |
| ITPR2 | inositol 1,4,5-triphosphase receptor, type 2 | Cytoplasm | 1.584 | x | | x | |
| PSMA2 | proteasome (prosome, macropain) subunit, alpha type, 2 | Cytoplasm | 1.563 | | x | | |
| PEA15 | phosphoprotein enriched in astrocytes 15 | Cytoplasm | 1.552 | | x | | |
| PPIA | peptidylprolyl isomerase A (cyclophilin A) | Cytoplasm | 1.546 | x | x | | x |
| YME1L1 | YME1-like 1 (S. cerevisiae) | Cytoplasm | 1.536 | x | | x | |
| NKTR | natural killer-tumor recognition sequence | Plasma Membrane | 1.522 | x | | x | |
| M6PR | mannose-6-phosphate receptor (cation dependent) | Cytoplasm | 1.513 | x | | | |
| ROD1 | ROD1 regulator of differentiation 1 (S. pombe) | Nucleus | 1.508 | x | | | |
| ANKIB1 | ankyrin repeat and IBR domain containing 1 | Nucleus | 1.448 | x | | x | |
| ABCB7 | ATP-binding [symbol] sub-family B (MDR/TAP) 7 | Cytoplasm | 1.444 | x | | x | |
| ATP2B1 | ATPase, Ca++ transporting, plasma membrane 1 | Plasma Membrane | 1.415 | x | | | |
| SETX | senataion | Nucleus | 1.409 | x | | x | |
| HNRNPU | homogeneous unclear ribonucleoprotein U | Nucleus | 1.402 | | x | | |
| TNRC68 | tri [symbol] repeat containing 68 | Unknown | 1.3 [symbol] | x | | x | |
| GDI2 | GDP disociation inhibitor 2 | Cytoplasm | 1.323 | x | x | | |
| PPHUN1 | [symbol] 1 | Nucleus | 1.2 [symbol] 1 | x | | x | |
| SF3B1 | splicing factor 3D, subunit 1, 155 kDa | Nucleus | 1.274 | x | | x | |
| TRIP4 | thyroid ho [symbol] one receptor interactor 4 | Nucleus | 1.2 [symbol] | x | | x | |
| NR3C1 | nuclear neceptor subfamily 3, group C1 | Nucleus | 1.253 | x | | x | |
| TBCB | tubulin folding colactor B | Cytoplasm | 1.227 | | x | | |
| PFDN2 | pre [symbol] subunit 2 | Cytoplasm | 1.225 | | x | | |
| PRDM4 | PR domain containing 4 | Nucleus | 1.191 | x | | x | |
| RGS3 | regulator of G-protein signaling 3 | Nucleus | −1.216 | x | | | |
| TUBB2C | tubulin, beta 2C | Cytoplasm | −1.241 | x | x | x | |
| HGS | hepatocyte growth factor-regulated subtrate | Cytoplasm | −1.309 | x | | x | |
| PTK2B | PTK2B protein tyrosine kinase 2 beta | Cytoplasm | −1.343 | x | | x | |
| CRTC2 | CREB regulated transcription coactivator 2 | Nucleus | −1.356 | x | | | |
| ARSA | arylsulfatase A | Cytoplasm | −1.389 | x | | x | |
| GTP8P1 | GTP binding protein 1 | Cytoplasm | −1.397 | x | | | |
| ADAMTS7 | ADAM metallopeptidase with thrombospondin type 1, 7 | Extracellular Space | −1.496 | x | | x | |
| RAB11B | RAB11B, member RAS oncogene family | Cytoplasm | −1.564 | x | | | |
| PPP1CB | protein phosphatase 1, catalytic subunit, beta isoform | Cytoplasm | −1.597 | x | | x | |
| FBXO38 | F-box protein 38 | Nucleus | −1.627 | x | | x | |
| HLA-G | major histocompatibility complex, class I, G | Plasma Membrane | −1.802 | x | | | |
| PNPLA2 | [symbol] -like phospholipase domain containing 2 | Cytoplasm | −1.802 | x | | x | |
| SLC39A7 | solute carrier family 39 (zinc transporter), member 7 | Plasma Membrane | −1.803 | x | | x | |
| FN1 | fibronectin 1 | Plasma Membrane | −2.616 | x | x | x | |

Based on the interature available in the IPA database the cellular localization and detection in bodily fluids is indicated.
Fold change is represented as the difference in expression level when compared to normal.
B = blood;
BAL = [symbol] Lavage Fluid:
P/S = Plasma/Serum:
SP = Sputum. [symbol]
indicates data missing or illegible when filed

TABLE 5
Unique Candidate Biomarkers for symptomatic disease when compared to healthy controls

| Gene | | | Fold | | | | |
|---|---|---|---|---|---|---|---|
| Symbol | Gene Description | Location | Change | B | BAL | P/S | Sp |
| PAEP | progestagen-associated endometrial protein | Extracellular Space | 2.492 | x | | x | |
| HOOK3 | hook homolog 3 (Drosophila) | Cytoplasm | 2.078 | x | | | |
| FAM13A1 | family with sequence similarity 13, member A1 | Unknown | 1.998 | x | | x | |
| RHCG | Rh family, C glycoprotein | Plasma Membrane | 1.903 | x | | | |
| HLA-DRA | major histocompatibility complex, class II, DR alpha | Plasma Membrane | 1.871 | x | x | | |
| IREB2 | iron-responsive element binding protein 2 | Cytoplasm | 1.864 | x | | x | |
| CLINT1 | clathrin interactor 1 | Cytoplasm | 1.845 | x | | x | |
| CRIP1 | cysteine-rich protein 1 (intestinal) | Cytoplasm | 1.773 | | x | | |
| PRKCI | protein kinase C, iota | Cytoplasm | 1.756 | x | | x | |
| BDP1 | B double prime 1, subunit of transcription Factor IIIB | Nucleus | 1.705 | x | x | x | |
| MAN2A1 | mannosidase, alpha, class 2A, member 1 | Cytoplasm | 1.678 | x | | | |
| SLC16A6 | solute carrier family 16, member 6 | Plasma Membrane | 1.657 | x | | x | |
| TNFAIP3 | tumor necrosis factor, alpha-induced protein 3 | Nucleus | 1.644 | x | | | |
| HLA-DOA | major histocompatibility complex, class II, DO alpha | Plasma Membrane | 1.643 | x | | | |
| MLL | myeloid/lymphoid or mixed-lineage leukemia | Nucleus | 1.625 | x | | x | |
| CYSLTR1 | cysteinyl leukotriene receptor 1 | Plasma Membrane | 1.615 | x | | | |
| MEF2C | myocyte enhancer factor 2C | Nucleus | 1.606 | x | | | |
| UBE3A | ubiquitin protein ligase E3A (Angelman syndrome) | Nucleus | 1.605 | x | | x | |
| DZIP3 | DAZ interacting protein 3, zinc finger | Cytoplasm | 1.593 | x | | x | |
| RPL10L | ribosomal protein L10-like | Nucleus | 1.589 | x | | x | |
| ITPR2 | inositol 1,4,5-triphosphate receptor, type 2 | Cytoplasm | 1.584 | x | | x | |
| PSMA2 | proteasome (prosome, macropain) subunit, alpha type, 2 | Cytoplasm | 1.563 | | x | | |
| PEA15 | phosphoprotein enriched in astrocytes 15 | Cytoplasm | 1.552 | | x | | |
| PPIA | peptidylprolyl isomerase A (cycloph [symbol] n A) | Cytoplasm | 1.546 | x | x | | x |
| YME1L1 | YME1-like 1 (S. cerevisiae) | Cytoplasm | 1.536 | x | | x | |
| NKTR | natural killer-tumor recognition sequence | Plasma Membrane | 1.522 | x | | x | |
| M6PR | mannose-6-phosphate receptor (cation dependent) | Cytoplasm | 1.613 | x | | | |
| ROD1 | ROD1 regulator of differentiation 1 (S. pombe) | Nucleus | 1.508 | x | | | |
| ANKIB1 | ankyrin repeat and IBR domain containing 1 | Nucleus | 1.448 | x | | x | |
| ABCB7 | ATP-binding c [symbol] sub-family B (MDR/TAP) 7 | Cytoplasm | 1.444 | x | | x | |
| ATP2B1 | ATPase, Ca++ transporting, plasma membrane 1 | Plasma Membrane | 1.415 | x | | | |
| SETX | senataxin | Nucleus | 1.409 | x | | x | |
| HNRNPU | heterogeneous nuclear ribonucleoprotein U | Nucleus | 1.402 | | x | | |
| TNRC68 | trinucleotide repeat containing 68 | Unknown | 1.366 | x | | x | |
| GDI2 | GDP dissociation inhibitor 2 | Cytoplasm | 1.323 | x | x | | |
| PPHLN1 | p [symbol] ph [symbol] n 1 | Nucleus | 1.281 | x | | x | |
| SF3B1 | splicing factor 3b, subunit 1, 155 kDa | Nucleus | 1.274 | x | | x | |
| TRIP4 | thyroid hormone receptor interactor 4 | Nucleus | 1.268 | x | | x | |
| NR3C1 | nuclear receptor subfamily 3, group C1 | Nucleus | 1.253 | x | | x | |
| TBCB | tubulin folding cofactor B | Cytoplasm | 1.227 | | x | | |
| PFDN2 | prefoldin subunit 2 | Cytoplasm | 1.225 | | x | | |
| PRDM4 | PR domain containing 4 | Nucleus | 1.191 | x | | x | |
| RGS3 | regulator of G-protein signaling 3 | Nucleus | −1.210 | x | | | |
| TUBB2C | tubulin, beta 2C | Cytoplasm | −1.241 | x | x | x | |
| HGS | hepatocyte growth factor-regulated substrate | Cytoplasm | −1.309 | x | | x | |
| PTK2B | PTK2B protein tyrosine kinase 2 beta | Cytoplasm | −1.343 | x | | x | |
| CRTC2 | CREB regulated transcription coactivator 2 | Nucleus | −1.356 | x | | | |
| ARSA | arylsulfatase A | Cytoplasm | −1.389 | x | | x | |
| GTPBP1 | GTP binding protein 1 | Cytoplasm | −1.397 | x | | | |
| ADAMTS7 | ADAM metallopeptidase with thrombospondin type 1, 7 | Extracellular Space | −1.496 | x | | x | |
| RAB11B | RAB11B, member RAS oncogene family | Cytoplasm | −1.564 | x | | | |
| PPP1CB | protein phosphatase 1, catalytic subunit, beta isoform | Cytoplasm | −1.597 | x | | x | |
| FBXO38 | F-box protein 38 | Nucleus | −1.627 | x | | x | |
| HLA-G | major histocompatibility complex, class I, G | Plasma Membrane | −1.802 | x | | | |
| PNPLA2 | patatin-like phospholipase domain containing 2 | Cytoplasm | −1.802 | x | | x | |
| SLC39A7 | solute carrier family 39 (zinc transporter), member 7 | Plasma Membrane | −1.803 | x | | x | |
| FN1 | fibronectin 1 | Plasma Membrane | −2.616 | x | x | x | |

Based on the interature available in the IPA database the cellular localization and detection in bod [symbol] y fluids is indicated.
Fold change is represented as the difference in expression level when compared to normal
B = blood;
BAL = Branchoalveolar Lavage Fluid;
P/S = Plasma/Serum;
SP = Sputum. [symbol]
indicates data missing or illegible when filed

TABLE 6A
Clinical and demographic IPF variables categorized by FVC.
A.

| | | Mild IPF | Severe IPF | Controls |
|---|---|---|---|---|
| Variable | Characteristics | (N = 27) | (N = 13) | (N = 31) |
| % Predicted | | 85.0 ± 8.1 | 42.5 ± 6.6 | NR |
| FVC | | | | |
| Age | Mean ± SD | 69.8 ± 8.4 | 65.3 ± 12.7 | 59.5 ± 13.5 |
| Sex | Male/Female | 19/8 | 10/3 | 13/18 |
| Smoking | Current | 0 | 0 | 4 |
| Status | Former | 7 | 7 | 14 |
| | Never | 18 | 6 | 13 |
| | Not Reported | 2 | 0 | 0 |
| Diagnosis | | IPF | IPF | Normal |

TABLE 6B
Clinical and demographic IPF variables categorized by D_{L}CO.
B.

| | | Mild IPF | Severe IPF | Controls |
|---|---|---|---|---|
| Variable | Characteristics | (N = 16) | (N = 15) | (N = 31) |
| % Predicted | | 77.1 ± 11.9 | 27.4 ± 5.3 | NR |
| D_{L}CO | | | | |
| Age | Mean ± SD | 67.4 ± 6.0 | 66.8 ± 13.7 | 59.5 ± 13.5 |
| Sex | Male/Female | 11/5 | 11/4 | 13/18 |
| Smoking | Current | 0 | 0 | 4 |
| Status | Former | 7 | 10 | 14 |
| | Never | 8 | 5 | 13 |
| | Not Reported | 1 | 0 | 0 |
| Diagnosis | | IPF | IPF | Normal |

TABLE 7
Peripheral blood cohort for percent predicted forced vital capacity (FVC).

| | Clinical | % Predicted | |
|---|---|---|---|
| ID | Diagnosis | FVC | Onset |
| MFVC01 | IPF | 76 | Mild |
| MFVC02 | IPF | 76 | Mild |
| MFVC03 | IPF | 77 | Mild |
| MFVC04 | IPF | 77 | Mild |
| MFVC05 | IPF | 77 | Mild |
| MFVC06 | IPF | 79 | Mild |
| MFVC07 | IPF | 80 | Mild |
| MFVC08 | IPF | 80 | Mild |
| MFVC09 | IPF | 81 | Mild |
| MFVC10 | IPF | 81 | Mild |
| MFVC11 | IPF | 81 | Mild |
| MFVC12 | IPF | 81 | Mild |
| MFVC13 | IPF | 82 | Mild |
| MFVC14 | IPF | 83 | Mild |
| MFVC15 | IPF | 83 | Mild |
| MFVC16 | IPF | 84 | Mild |
| MFVC17 | IPF | 86 | Mild |
| MFVC18 | IPF | 86 | Mild |
| MFVC19 | IPF | 87 | Mild |
| MFVC20 | IPF | 90 | Mild |
| MFVC21 | IPF | 91 | Mild |
| MFVC22 | IPF | 91 | Mild |
| MFVC23 | IPF | 92 | Mild |
| MFVC24 | IPF | 94 | Mild |
| MFVC25 | IPF | 101 | Mild |
| MFVC26 | IPF | 111 | Mild |
| MFVC27 | IPF | 88 | Mild |
| SFVC01 | IPF | 26 | Severe |
| SFVC02 | IPF | 37 | Severe |
| SFVC03 | IPF | 37 | Severe |
| SFVC04 | IPF | 41 | Severe |
| SFVC05 | IPF | 42 | Severe |
| SFVC06 | IPF | 43 | Severe |
| SFVC07 | IPF | 43 | Severe |
| SFVC08 | IPF | 44 | Severe |
| SFVC09 | IPF | 45 | Severe |
| SFVC10 | IPF | 45 | Severe |
| SFVC11 | IPF | 50 | Severe |
| SFVC12 | IPF | 50 | Severe |
| SFVC13 | IPF | 50 | Severe |

TABLE 8
Peripheral blood cohort for percent predicted diffusion lung capacity for
carbon monoxide (D_{L}CO).

| | | % | |
|---|---|---|---|
| | Clinical | Predicted | |
| ID | Diagnosis | D_{L}CO | Onset |
| MDLCO01 | IPF | 65 | Mild |
| MDLCO02 | IPF | 65 | Mild |
| MDLCO03 | IPF | 66 | Mild |
| MDLCO04 | IPF | 66 | Mild |
| MDLCO05 | IPF | 66 | Mild |
| MDLCO06 | IPF | 69 | Mild |
| MDLCO07 | IPF | 71 | Mild |
| MDLCO08 | IPF | 75 | Mild |
| MDLCO09 | IPF | 77 | Mild |
| MDLCO10 | IPF | 78 | Mild |
| MDLCO11 | IPF | 79 | Mild |
| MDLCO12 | IPF | 83 | Mild |
| MDLCO13 | IPF | 85 | Mild |
| MDLCO14 | IPF | 87 | Mild |
| MDLCO15 | IPF | 99 | Mild |
| MDLCO16 | IPF | 103 | Mild |
| SDLCO01 | IPF | 18 | Severe |
| SDLCO02 | IPF | 19 | Severe |
| SDLCO03 | IPF | 21 | Severe |
| SDLCO04 | IPF | 24 | Severe |
| SDLCO05 | IPF | 24 | Severe |
| SDLCO06 | IPF | 25 | Severe |
| SDLCO07 | IPF | 28 | Severe |
| SDLCO08 | IPF | 29 | Severe |
| SDLCO09 | IPF | 30 | Severe |
| SDLCO10 | IPF | 30 | Severe |
| SDLCO11 | IPF | 31 | Severe |
| SDLCO12 | IPF | 32 | Severe |
| SDLCO13 | IPF | 32 | Severe |
| SDLCO14 | IPF | 34 | Severe |
| SDLCO15 | IPF | 34 | Severe |

TABLE 9
Differentially expressed transcripts between mild and severe cases of IPF.

| | Entrez Gene | | Accession | Fold | | |
|---|---|---|---|---|---|---|
| Symbol | Name | Probe ID | Number | Change | Location | Type(s) |
| CAMP | cathelicidin | A_23_P253791 | NM_004345 | 2.591 | Cytoplasm | other |
| | antimicrobial | | | | | |
| | peptide | | | | | |
| CEACAM6 | carcinoembryonic | A_23_P421483 | BC005008 | 2.353 | Plasma | other |
| (includes | antigen-related | | | | Membrane | |
| EG: 4680) | cell adhesion | | | | | |
| | molecule 6 | | | | | |
| CTSG | cathepsin G | A_23_P140384 | NM_001911 | 2.703 | Cytoplasm | peptidase |
| DEFA3 | defensin, alpha3, | A_23_P31816 | NM_005217 | 2.379 | Extracellular | other |
| (includes | neutrophil-specific | | | | Space | |
| EG: 1668) | | | | | | |
| DEFA4 | defensin, alpha 4, | A_23_P326080 | NM_001925 | 3.713 | Extracellular | other |
| (includes | corticostatin | | | | Space | |
| EG: 1669) | | | | | | |
| OLFM4 | olfactomedin 4 | A_24_P181254 | NM_006418 | 3.807 | unknown | other |
| HLTF | helicase-like | A32_P210798 | BF513730 | 1.413 | unknown | unknown |
| | transcription factor | | | | | |
| PACSIN1 | protein kinase C | A_23_P258088 | NM_020804 | −1.511 | Cytoplasm | kinase |
| | and casein kinase | | | | | |
| | substrate in | | | | | |
| | neurons 1 | | | | | |
| FLJ11710 | hypothetical | A_23_P3921 | AK021772 | −1.798 | unknown | other |
| | protein FLJ11710 | | | | | |
| GABBR1 | gamma- | A_23_P93302 | NM_001470 | −1.471 | Plasma | G-protein |
| | aminobutyric acid | | | | Membrane | coupled |
| | (GABA) B | | | | | receptor |
| | receptor, 1 | | | | | |
| IGHM | immunoglobulin | A_24_P417352 | BX161420 | −2.451 | Plasma | transmembrane |
| | heavy constant | | | | Membrane | receptor |
| | mu | | | | | |
| unknown | unknown | A_23_P91743 | unknown | −1.884 | unknown | unknown |
| unknown | unknown | A_24_P481375 | AK021668 | −1.706 | unknown | unknown |

TABLE 10
p-value ranges for associated network bio-functions.

| Function | p-Value Range | # of Molecules |
|---|---|---|
| Inflammatory | 1.79E\hat{ }−4-3.94E\hat{ }−2 | 4 |
| Response | | |
| Cellular Movement | 9.39E\hat{ }−5-3.94E\hat{ }−2 | 3 |
| Immune Trafficking | 1.23E\hat{ }−4-3.94E\hat{ }−2 | 4 |
| Genetic disorder | 1.04E\hat{ }−3-4.29E\hat{ }−2 | 4 |
| Cell-to-cell signaling | 6.07E\hat{ }−4-4.17E\hat{ }−2 | 5 |

TABLE 11
Fold-changes in candidate biomarkers to monitor IPF disease progression.

| | | | Normal vs. |
|---|---|---|---|
| Symbol | Normal vs. Mild IPF | Mild vs. Severe IPF | Severe IPF |
| DEFA3 | 1.465 | 2.379 | 3.485 |
| FLJ11710 | −1.455 | −1.798 | −2.024 |
| CEACAM6 | NDE | 2.353 | 2.436 |
| CAMP | NDE | 2.591 | 2.837 |
| CTSG | NDE | 2.703 | 2.899 |
| DEFA4 | NDE | 3.713 | 3.277 |
| OLFM4 | NDE | 3.807 | 3.914 |
| HLTF | NDE | 1.413 | −1.208 |
| PACSIN1 | NDE | −1.511 | −1.377 |
| GABBR1 | NDE | −1.471 | −1.391 |
| IGHM | NDE | −2.451 | −3.148 |

TABLE 12
Differentially expressed transcripts for mild IPF vs normal controls

| | | | | | Fold- |
|---|---|---|---|---|---|
| Probe | AccNum | Symbol | Description | Gene Title | Change |
| A_32_P166272 | NA | NA | NA | NA | 11.705 |
| A_24_P297078 | NM_020531 | C20orf3 | chromosome 20 | chromosome 20 | 6.037 |
| | | | open reading frame 3 | open reading frame 3 | |
| A_24_P134816 | NM_182557 | BCL9L | B-cell | B-cell CLL/lymphoma | 3.170 |
| | | | CLL/lymphoma 9- | 9-like | |
| | | | like | | |
| A_24_P252996 | NM_000804 | FOLR3 | folate receptor 3 | folate receptor 3 | 3.004 |
| | | | (gamma) | (gamma) | |
| A_23_P79398 | NM_004633 | IL1R2 | interleukin 1 | interleukin 1 | 2.534 |
| | | | receptor, type II | receptor, type II | |
| A_23_P4283 | NM_017523 | XAF1 | XIAP associated | XIAP associated | 2.362 |
| | | | factor 1 | factor-1 | |
| A_24_P263793 | NM_002003 | FCN1 | ficolin | ficolin | 2.259 |
| | | | (collagen/fibrinogen | (collagen/fibrinogen | |
| | | | domain containing) 1 | domain containing) 1 | |
| A_23_P4286 | NM_017523 | XAF1 | XIAP associated | XIAP associated | 2.247 |
| | | | factor 1 | factor-1 | |
| A_23_P40174 | NM_004994 | MMP9 | matrix | matrix | 2.242 |
| | | | metallopeptidase 9 | metalloproteinase 9 | |
| | | | (gelatinase B, | (gelatinase B, 92 kDa | |
| | | | 92 kDa gelatinase, | gelatinase, 92 kDa | |
| | | | 92 kDa type IV | type IV collagenase) | |
| | | | collagenase) | | |
| A_23_P49708 | NM_002087 | GRN | granulin | granulin | 2.237 |
| A_24_P504621 | AA707467 | NA | NA | NA | 2.225 |
| A_23_P39925 | NM_003494 | DYSF | dysferlin, limb | NA | 2.223 |
| | | | girdle muscular | | |
| | | | dystrophy 2B | | |
| | | | (autosomal | | |
| | | | recessive) | | |
| A_32_P234459 | NR_001434 | HLA-H | major | NA | 2.221 |
| | | | histocompatibility | | |
| | | | complex, class I, H | | |
| | | | (pseudogene) | | |
| A_24_P10233 | NM_014326 | DAPK2 | death-associated | death-associated | 2.219 |
| | | | protein kinase 2 | protein kinase 2 | |
| A_23_P157875 | NM_002003 | FCN1 | ficolin | NA | 2.198 |
| | | | (collagen/fibrinogen | | |
| | | | domain containing) 1 | | |
| A_23_P4096 | NM_000717 | CA4 | carbonic anhydrase | carbonic anhydrase | 2.177 |
| | | | IV | IV | |
| A_24_P81740 | NM_006755 | TALDO1 | transaldolase 1 | transaldolase 1 | 2.163 |
| A_23_P27584 | NM_001020818 | MYADM | myeloid-associated | myeloid-associated | 2.135 |
| | | | differentiation | differentiation marker | |
| | | | marker | | |
| A_24_P161933 | CR608347 | HLA-B | major | major | 2.125 |
| | | | histocompatibility | histocompatibility | |
| | | | complex, class I, B | complex, class I, B | |
| A_23_P142750 | NM_002759 | EIF2AK2 | eukaryotic | eukaryotic translation | 2.123 |
| | | | translation initiation | initiation factor 2- | |
| | | | factor 2-alpha | alpha kinase 2 | |
| | | | kinase 2 | | |
| A_23_P77807 | NM_030665 | RAI1 | retinoic acid | retinoic acid induced 1 | 2.122 |
| | | | induced 1 | | |
| A_24_P390668 | NM_005892 | FMNL1 | formin-like 1 | formin-like 1 | 2.111 |
| A_23_P139786 | NM_003733 | OASL | 2′-5′-oligoadenylate | 2′-5′-oligoadenylate | 2.098 |
| | | | synthetase-like | synthetase-like | |
| A_23_P12680 | NM_001042465 | PSAP | prosaposin | prosaposin (variant | 2.083 |
| | | | | Gaucher disease and | |
| | | | | variant | |
| | | | | metachromatic | |
| | | | | leukodystrophy) | |
| A_24_P283189 | NM_000591 | CD14 | CD14 molecule | CD14 antigen | 2.081 |
| A_24_P309317 | NM_001042465 | PSAP | prosaposin | prosaposin (variant | 2.080 |
| | | | | Gaucher disease and | |
| | | | | variant | |
| | | | | metachromatic | |
| | | | | leukodystrophy) | |
| A_23_P122863 | NM_001001555 | GRB10 | growth factor | NA | 2.078 |
| | | | receptor-bound | | |
| | | | protein 10 | | |
| A_23_P157879 | NM_002003 | FCN1 | ficolin | ficolin | 2.072 |
| | | | (collagen/fibrinogen | (collagen/fibrinogen | |
| | | | domain containing) 1 | domain containing) 1 | |
| A_23_P44993 | NM_006755 | TALDO1 | transaldolase 1 | transaldolase 1 | 2.071 |
| A_32_P70158 | NM_006864 | LILRB3 | leukocyte | NA | 2.069 |
| | | | immunoglobulin- | | |
| | | | like receptor, | | |
| | | | subfamily B (with | | |
| | | | TM and ITIM | | |
| | | | domains), member 3 | | |
| A_24_P123616 | NM_005345 | HSPA1A | heat shock 70 kDa | heat shock 70 kDa | 2.068 |
| | | | protein 1A | protein 1A | |
| A_24_P88690 | NM_000578 | SLC11A1 | solute carrier family | solute carrier family | 2.051 |
| | | | 11 (proton-coupled | 11 (proton-coupled | |
| | | | divalent metal ion | divalent metal ion | |
| | | | transporters), | transporters), | |
| | | | member 1 | member 1 | |
| A_23_P135755 | NM_001557 | CXCR2 | chemokine (C-X-C | interleukin 8 | 2.046 |
| | | | motif) receptor 2 | receptor, beta | |
| A_24_P89701 | NM_000883 | IMPDH1 | IMP (inosine | IMP (inosine | 2.045 |
| | | | monophosphate) | monophosphate) | |
| | | | dehydrogenase 1 | dehydrogenase 1 | |
| A_24_P682285 | NM_005345 | HSPA1A | heat shock 70 kDa | NA | 2.042 |
| | | | protein 1A | | |
| A_23_P4662 | NM_005178 | BCL3 | B-cell | B-cell CLL/lymphoma 3 | 2.036 |
| | | | CLL/lymphoma 3 | | |
| A_24_P101771 | NA | NA | NA | NA | 2.030 |
| A_23_P325438 | NM_015171 | XPO6 | exportin 6 | exportin 6 | 2.019 |
| A_23_P873 | BC031655 | C1orf38 | chromosome 1 | chromosome 1 open | 2.013 |
| | | | open reading frame | reading frame 38 | |
| | | | 38 | | |
| A_23_P26865 | NM_002470 | MYH3 | myosin, heavy | myosin, heavy | 2.011 |
| | | | chain 3, skeletal | polypeptide 3, | |
| | | | muscle, embryonic | skeletal muscle, | |
| | | | | embryonic | |
| A_32_P203154 | NM_000982 | RPL21 | ribosomal protein | NA | 0.500 |
| | | | L21 | | |
| A_23_P63953 | XM_929084 | NA | NA | NA | 0.499 |
| A_24_P50554 | XR_018405 | NA | NA | NA | 0.499 |
| A_24_P84808 | XR_015548 | NA | NA | NA | 0.497 |
| A_24_P57898 | NM_080606 | BHLHE23 | basic helix-loop- | NA | 0.496 |
| | | | helix family, | | |
| | | | member e23 | | |
| A_24_P572229 | NA | NA | NA | NA | 0.496 |
| A_32_P10424 | AX721252 | NA | NA | NA | 0.496 |
| A_24_P76120 | NA | NA | NA | NA | 0.496 |
| A_24_P41662 | NA | NA | NA | NA | 0.495 |
| A_24_P101271 | NA | NA | NA | NA | 0.494 |
| A_24_P213375 | NA | NA | NA | NA | 0.493 |
| A_24_P375949 | XR_019375 | NA | NA | NA | 0.493 |
| A_24_P298604 | XR_015536 | NA | NA | NA | 0.493 |
| A_24_P366546 | XR_018695 | NA | NA | NA | 0.491 |
| A_32_P74615 | NM_001003845 | SP5 | Sp5 transcription | NA | 0.490 |
| | | | factor | | |
| A_24_P789842 | NA | NA | NA | NA | 0.490 |
| A_24_P136905 | AF116713 | NA | NA | inter-alpha (globulin) | 0.490 |
| | | | | inhibitor H1 | |
| A_24_P409681 | NA | NA | NA | NA | 0.489 |
| A_24_P34575 | NM_006236 | POU3F3 | POU class 3 | POU domain, class | 0.489 |
| | | | homeobox 3 | 3, transcription factor 3 | |
| A_23_P56736 | NM_080386 | TUBA3D | tubulin, alpha 3d | alpha-tubulin isotype | 0.489 |
| | | | | H2-alpha | |
| A_24_P178693 | XR_018303 | NA | NA | NA | 0.489 |
| A_32_P234738 | NM_000982 | RPL21 | ribosomal protein | ribosomal protein | 0.488 |
| | | | L21 | L21 | |
| A_24_P84408 | NA | NA | NA | NA | 0.488 |
| A_24_P298238 | NA | NA | NA | NA | 0.488 |
| A_24_P419028 | AB014771 | MOP-1 | MOP-1 | RasGEF domain | 0.487 |
| | | | | family, member 1B | |
| A_32_P186981 | NM_000985 | RPL17 | ribosomal protein | ribosomal protein | 0.487 |
| | | | L17 | L17 | |
| A_23_P323685 | NM_003543 | HIST1H4H | histone cluster 1, | histone 1, H4h | 0.485 |
| | | | H4h | | |
| A_23_P50834 | NM_182515 | ZNF714 | zinc finger protein | hypothetical protein | 0.485 |
| | | | 714 | LOC148206 | |
| A_24_P392082 | NA | NA | NA | NA | 0.482 |
| A_24_P542291 | XR_017668 | LOC339352 | similar to Putative | hypothetical | 0.481 |
| | | | ATP-binding | LOC339352 | |
| | | | domain-containing | | |
| | | | protein 3-like | | |
| | | | protein | | |
| A_23_P416314 | BC034222 | HRASLS5 | HRAS-like | H-rev107-like protein 5 | 0.480 |
| | | | suppressor family, | | |
| | | | member 5 | | |
| A_24_P918810 | XR_018482 | NA | NA | NA | 0.479 |
| A_24_P848662 | CR594528 | LOC100131582 | hypothetical protein | NA | 0.479 |
| | | | LOC100131582 | | |
| A_32_P98348 | AK097037 | ZNF525 | zinc finger protein | zinc finger protein | 0.478 |
| | | | 525 | 525 | |
| A_24_P340976 | XR_018155 | NA | NA | NA | 0.478 |
| A_24_P127621 | NA | NA | NA | NA | 0.477 |
| A_32_P128781 | NA | NA | NA | NA | 0.477 |
| A_24_P144275 | NA | NA | NA | NA | 0.474 |
| A_23_P315320 | NM_145659 | IL27 | interleukin 27 | interleukin 27 | 0.472 |
| A_24_P412734 | NM_173502 | PRSS36 | protease, serine, | protease, serine, 36 | 0.472 |
| | | | 36 | | |
| A_24_P237328 | NM_014507 | MCAT | malonyl CoA:ACP | malonyl-CoA:acyl | 0.472 |
| | | | acyltransferase | carrier protein | |
| | | | (mitochondrial) | transacylase, | |
| | | | | mitochondrial | |
| A_32_P34201 | XR_018643 | NA | NA | NA | 0.471 |
| A_24_P830667 | NM_000982 | RPL21 | ribosomal protein | NA | 0.470 |
| | | | L21 | | |
| A_24_P166407 | NM_003544 | HIST1H4B | histone cluster 1, | histone 1, H4b | 0.469 |
| | | | H4b | | |
| A_24_P203909 | NM_033625 | RPL34 | ribosomal protein | NA | 0.469 |
| | | | L34 | | |
| A_24_P714620 | NA | NA | NA | NA | 0.467 |
| A_24_P281304 | NA | NA | NA | NA | 0.467 |
| A_24_P126890 | NM_001024921 | RPL9 | ribosomal protein | NA | 0.466 |
| | | | L9 | | |
| A_24_P392713 | AK124741 | NA | NA | NA | 0.466 |
| A_24_P392195 | NA | NA | NA | NA | 0.466 |
| A_32_P88317 | NA | NA | NA | NA | 0.465 |
| A_24_P575336 | XR_017056 | NA | NA | NA | 0.465 |
| A_24_P366457 | NA | NA | NA | NA | 0.463 |
| A_24_P606663 | XR_017639 | NA | NA | NA | 0.463 |
| A_24_P169378 | NM_001011 | RPS7 | ribosomal protein | NA | 0.462 |
| | | | S7 | | |
| A_24_P57837 | NA | NA | NA | NA | 0.461 |
| A_32_P158746 | NM_000985 | RPL17 | ribosomal protein | NA | 0.461 |
| | | | L17 | | |
| A_24_P358205 | NA | NA | NA | NA | 0.461 |
| A_24_P213354 | XR_015710 | LOC729046 | similar to ribosomal | NA | 0.461 |
| | | | protein L17 | | |
| A_24_P264143 | XR_019235 | NA | NA | NA | 0.461 |
| A_24_P32836 | NA | NA | NA | NA | 0.461 |
| A_24_P349636 | XR_016879 | NA | NA | NA | 0.460 |
| A_24_P357518 | NM_000982 | RPL21 | ribosomal protein | NA | 0.460 |
| | | | L21 | | |
| A_24_P280803 | BC018140 | RPS21 | ribosomal protein | ribosomal protein | 0.459 |
| | | | S21 | S21 | |
| A_24_P47681 | NM_018448 | CAND1 | cullin-associated | TBP-interacting | 0.458 |
| | | | and neddylation- | protein | |
| | | | dissociated 1 | | |
| A_24_P144666 | XR_017247 | NA | NA | NA | 0.458 |
| A_24_P33213 | NA | NA | NA | NA | 0.457 |
| A_32_P203013 | BC030568 | RPS10P7 | ribosomal protein | hypothetical | 0.457 |
| | | | S10 pseudogene 7 | LOC376693 | |
| A_24_P375932 | NA | NA | NA | NA | 0.457 |
| A_24_P307443 | XR_018808 | NA | NA | NA | 0.456 |
| A_24_P93452 | NA | NA | NA | NA | 0.455 |
| A_24_P350008 | NA | NA | NA | NA | 0.454 |
| A_32_P58074 | NM_001006 | RPS3A | ribosomal protein | NA | 0.452 |
| | | | S3A | | |
| A_24_P367369 | NA | NA | NA | NA | 0.451 |
| A_24_P127312 | XR_019013 | NA | NA | NA | 0.451 |
| A_24_P324224 | NA | NA | NA | NA | 0.449 |
| A_24_P383999 | NM_001006 | RPS3A | ribosomal protein | NA | 0.448 |
| | | | S3A | | |
| A_32_P113742 | BC104478 | RPL21 | ribosomal protein | NA | 0.447 |
| | | | L21 | | |
| A_24_P367191 | XR_019544 | NA | NA | NA | 0.445 |
| A_24_P92661 | XR_019597 | NA | NA | NA | 0.442 |
| A_24_P117782 | NM_033129 | SCRT2 | scratch homolog 2, | NA | 0.442 |
| | | | zinc finger protein | | |
| | | | (Drosophila) | | |
| A_24_P384411 | NA | NA | NA | NA | 0.441 |
| A_23_P7229 | NM_033625 | RPL34 | ribosomal protein | ribosomal protein | 0.437 |
| | | | L34 | L34 | |
| A_24_P76358 | XR_018444 | NA | NA | NA | 0.436 |
| A_24_P307205 | XR_018138 | NA | NA | NA | 0.434 |
| A_24_P212726 | NA | NA | NA | NA | 0.432 |
| A_24_P212864 | XR_018048 | NA | NA | NA | 0.432 |
| A_24_P464798 | NA | NA | NA | NA | 0.432 |
| A_32_P145856 | NA | NA | NA | NA | 0.429 |
| A_24_P323698 | NA | NA | NA | NA | 0.429 |
| A_24_P917457 | XR_019532 | NA | NA | NA | 0.428 |
| A_24_P33607 | XR_019386 | NA | NA | NA | 0.427 |
| A_24_P685729 | NA | NA | NA | NA | 0.426 |
| A_24_P50437 | BC065737 | LOC100287512 | similar to ribosomal | NA | 0.426 |
| | | | protein S3a | | |
| A_32_P113154 | CR615245 | LOC100131581 | hypothetical | NA | 0.420 |
| | | | LOC100131581 | | |
| A_24_P755505 | NA | NA | NA | NA | 0.416 |
| A_24_P410070 | NA | NA | NA | NA | 0.415 |
| A_24_P41551 | XR_018025 | NA | NA | NA | 0.415 |
| A_32_P135818 | NM_001006 | RPS3A | ribosomal protein | NA | 0.415 |
| | | | S3A | | |
| A_24_P152753 | XR_019376 | NA | NA | NA | 0.413 |
| A_24_P367139 | NA | NA | NA | NA | 0.412 |
| A_23_P200955 | NA | NA | NA | NA | 0.406 |
| A_23_P29079 | NM_001002021 | NA | NA | phosphofructokinase, | 0.404 |
| | | | | liver | |
| A_32_P190648 | NA | NA | NA | interferon-related | 0.404 |
| | | | | developmental | |
| | | | | regulator 1 | |
| A_32_P155364 | NM_000971 | RPL7 | ribosomal protein | ribosomal protein L7 | 0.401 |
| | | | L7 | | |
| A_24_P367199 | NA | NA | NA | NA | 0.400 |
| A_32_P175580 | BC001697 | RPS15A | ribosomal protein | NA | 0.400 |
| | | | S15a | | |
| A_24_P135771 | NA | NA | NA | NA | 0.400 |
| A_24_P204474 | NA | NA | NA | NA | 0.399 |
| A_24_P289404 | NM_001029 | RPS26 | ribosomal protein | NA | 0.399 |
| | | | S26 | | |
| A_32_P100974 | NM_000986 | RPL24 | ribosomal protein | NA | 0.395 |
| | | | L24 | | |
| A_24_P110101 | NA | NA | NA | NA | 0.388 |
| A_24_P280897 | NA | NA | NA | NA | 0.386 |
| A_24_P675947 | NA | NA | NA | NA | 0.385 |
| A_24_P315326 | XR_016541 | NA | NA | NA | 0.378 |
| A_24_P306527 | NA | NA | NA | NA | 0.375 |
| A_24_P221375 | NA | NA | NA | NA | 0.374 |
| A_24_P112542 | NA | NA | NA | NA | 0.364 |
| A_24_P49597 | NA | NA | NA | NA | 0.355 |
| A_24_P878388 | NA | NA | NA | NA | 0.353 |
| A_24_P161494 | NA | NA | NA | NA | 0.328 |
| A_23_P69652 | NM_080819 | GPR78 | G protein-coupled | G protein-coupled | 0.312 |
| | | | receptor 78 | receptor 78 | |

TABLE 13
Differentially expressed transcripts for severe IPF vs normal controls

| | | | | | Fold- |
|---|---|---|---|---|---|
| Probe | AccNum | Symbol | Description | Gene Title | Change |
| A_24_P181254 | NM_006418 | OLFM4 | olfactomedin 4 | NA | 3.914 |
| A_23_P122863 | NM_001001555 | GRB10 | growth factor | NA | 3.608 |
| | | | receptor-bound | | |
| | | | protein 10 | | |
| A_23_P40174 | NM_004994 | MMP9 | matrix | matrix | 3.499 |
| | | | metallopeptidase 9 | metalloproteinase 9 | |
| | | | (gelatinase B, | (gelatinase B, | |
| | | | 92 kDa gelatinase, | 92 kDa gelatinase, | |
| | | | 92 kDa type IV | 92 kDa type IV | |
| | | | collagenase) | collagenase) | |
| A_23_P31816 | NM_005217 | DEFA3 | defensin, alpha 3, | defensin, alpha 1, | 3.485 |
| | | | neutrophil-specific | myeloid-related | |
| | | | | sequence | |
| A_23_P79398 | NM_004633 | IL1R2 | interleukin 1 | interleukin 1 | 3.399 |
| | | | receptor, type II | receptor, type II | |
| A_23_P326080 | NM_001925 | DEFA4 | defensin, alpha 4, | defensin, alpha 4, | 3.277 |
| | | | corticostatin | corticostatin | |
| A_23_P166848 | NM_002343 | LTF | lactotransferrin | lactotransferrin | 3.247 |
| A_23_P30707 | AK000385 | NA | NA | NA | 2.998 |
| A_23_P140384 | NM_001911 | CTSG | cathepsin G | cathepsin G | 2.899 |
| A_23_P253791 | NM_004345 | CAMP | cathelicidin | cathelicidin | 2.837 |
| | | | antimicrobial | antimicrobial | |
| | | | peptide | peptide | |
| A_23_P380240 | NM_001816 | CEACAM8 | carcinoembryonic | carcinoembryonic | 2.834 |
| | | | antigen-related cell | antigen-related cell | |
| | | | adhesion molecule 8 | adhesion molecule 8 | |
| A_23_P217269 | NM_007268 | VSIG4 | V-set and | V-set and | 2.810 |
| | | | immunoglobulin | immunoglobulin | |
| | | | domain containing 4 | domain containing 4 | |
| A_23_P111321 | NM_000045 | ARG1 | arginase, liver | arginase, liver | 2.683 |
| A_23_P111206 | NM_004117 | FKBP5 | FK506 binding | FK506 binding | 2.601 |
| | | | protein 5 | protein 5 | |
| A_24_P750164 | AK055877 | LOC151438 | hypothetical protein | hypothetical protein | 2.594 |
| | | | LOC151438 | LOC151438 | |
| A_23_P208747 | NM_005091 | PGLYRP1 | peptidoglycan | peptidoglycan | 2.559 |
| | | | recognition protein 1 | recognition protein 1 | |
| A_23_P4096 | NM_000717 | CA4 | carbonic anhydrase | carbonic anhydrase | 2.515 |
| | | | IV | IV | |
| A_23_P421483 | BC005008 | CEACAM6 | carcinoembryonic | carcinoembryonic | 2.436 |
| | | | antigen-related cell | antigen-related cell | |
| | | | adhesion molecule | adhesion molecule 5 | |
| | | | 6 (non-specific | | |
| | | | cross reacting | | |
| | | | antigen) | | |
| A_23_P169437 | NM_005564 | LCN2 | lipocalin 2 | lipocalin 2 | 2.425 |
| | | | | (oncogene 24p3) | |
| A_32_P128980 | BC062780 | NA | NA | NA | 2.397 |
| A_24_P233995 | NM_022746 | MOSC1 | MOCO sulphurase | hypothetical protein | 2.386 |
| | | | C-terminal domain | FLJ22390 | |
| | | | containing 1 | | |
| A_23_P71033 | NM_005338 | HIP1 | huntingtin | huntingtin | 2.386 |
| | | | interacting protein 1 | interacting protein 1 | |
| A_23_P348876 | AK022678 | NA | NA | NA | 2.385 |
| A_24_P206604 | NM_004566 | PFKFB3 | 6-phosphofructo-2- | 6-phosphofructo-2- | 2.315 |
| | | | kinase/fructose-2,6- | kinase/fructose-2,6- | |
| | | | biphosphatase 3 | biphosphatase 3 | |
| A_23_P216094 | NM_004318 | ASPH | aspartate beta- | aspartate beta- | 2.291 |
| | | | hydroxylase | hydroxylase | |
| A_23_P206760 | NM_005143 | HP | haptoglobin | haptoglobin | 2.260 |
| A_23_P153741 | NM_001700 | AZU1 | azurocidin 1 | azurocidin 1 | 2.228 |
| | | | | (cationic | |
| | | | | antimicrobial | |
| | | | | protein 37) | |
| A_23_P8640 | NM_001039966 | GPER | G protein-coupled | G protein-coupled | 2.173 |
| | | | estrogen receptor 1 | receptor 30 | |
| A_24_P89257 | NM_001031711 | ERGIC1 | endoplasmic | endoplasmic | 2.150 |
| | | | reticulum-golgi | reticulum-golgi | |
| | | | intermediate | intermediate | |
| | | | compartment | compartment 32 kDa | |
| | | | (ERGIC) 1 | protein | |
| A_23_P90041 | NM_033297 | NLRP12 | NLR family, pyrin | NACHT, leucine | 2.150 |
| | | | domain containing | rich repeat and | |
| | | | 12 | PYD containing 12 | |
| A_23_P39925 | NM_003494 | DYSF | dysferlin, limb girdle | NA | 2.096 |
| | | | muscular dystrophy | | |
| | | | 28 (autosomal | | |
| | | | recessive) | | |
| A_23_P130961 | NM_001972 | ELANE | elastase, neutrophil | elastase 2, | 2.081 |
| | | | expressed | neutrophil | |
| A_32_P902957 | NM_138450 | ARL11 | ADP-ribosylation | ADP-ribosylation | 2.081 |
| | | | factor-like 11 | factor-like 11 | |
| A_24_P186370 | NM_002444 | MSN | moesin | moesin | 2.063 |
| A_24_P338603 | NM_003036 | SKI | v-ski sarcoma viral | NA | 2.051 |
| | | | oncogene homolog | | |
| | | | (avian) | | |
| A_24_P116669 | NM_138793 | CANT1 | calcium activated | calcium activated | 2.046 |
| | | | nucleotidase 1 | nucleotidase 1 | |
| A_24_P418203 | NM_033655 | CNTNAP3 | contactin | contactin | 2.039 |
| | | | associated protein- | associated protein- | |
| | | | like 3 | like 3 | |
| A_23_P330561 | NM_174918 | C19orf59 | chromosome 19 | NA | 2.020 |
| | | | open reading frame | | |
| | | | 59 | | |
| A_23_P48676 | NM_002863 | PYGL | phosphorylase, | phosphorylase, | 2.000 |
| | | | glycogen, liver | glycogen; liver | |
| | | | | (Hers disease, | |
| | | | | glycogen storage | |
| | | | | disease type VI) | |
| A_23_P371076 | NA | NA | NA | Kruppel-like factor | 0.500 |
| | | | | 12 | |
| A_23_P126844 | NM_148965 | TNFRSF25 | tumor necrosis | tumor necrosis | 0.499 |
| | | | factor receptor | factor receptor | |
| | | | superfamily, | superfamily, | |
| | | | member 25 | member 25 | |
| A_24_P37020 | NA | NA | NA | NA | 0.498 |
| A_32_P71796 | NA | NA | NA | small EDRK-rich | 0.498 |
| | | | | factor 1A | |
| | | | | (telomeric) | |
| A_32_P173744 | CR603215 | hCG_17955 | high-mobility group | NA | 0.495 |
| | | | nucleosome | | |
| | | | binding domain 1 | | |
| | | | pseudogene | | |
| A_23_P39067 | NM_003121 | SPIB | Spi-B transcription | Spi-B transcription | 0.495 |
| | | | factor (Spi-1/PU.1 | factor (Spi-1/PU.1 | |
| | | | related) | related) | |
| A_23_P3921 | AK021772 | FLJ11710 | hypothetical protein | NA | 0.494 |
| | | | FLJ11710 | | |
| A_24_P24142 | XR_019250 | NA | NA | NA | 0.494 |
| A_24_P409402 | XR_016530 | NA | NA | NA | 0.492 |
| A_24_P418536 | XR_016540 | NA | NA | NA | 0.490 |
| A_24_P621701 | NA | NA | NA | NA | 0.490 |
| A_24_P204474 | NA | NA | NA | NA | 0.490 |
| A_24_P264143 | XR_019235 | NA | NA | NA | 0.490 |
| A_32_P8813 | AK090515 | LOC283663 | hypothetical | hypothetical protein | 0.489 |
| | | | LOC283663 | LOC283663 | |
| A_23_P207201 | NM_001039933 | CD79B | CD79b molecule, | CD79B antigen | 0.487 |
| | | | immunoglobulin- | (immunoglobulin- | |
| | | | associated beta | associated beta) | |
| A_24_P178693 | XR_018303 | NA | NA | NA | 0.486 |
| A_24_P713185 | NA | NA | NA | NA | 0.485 |
| A_24_P367399 | NA | NA | NA | NA | 0.485 |
| A_24_P340976 | XR_018155 | NA | NA | NA | 0.480 |
| A_23_P113572 | NM_001770 | CD19 | CD19 molecule | NA | 0.479 |
| A_24_P144163 | NA | NA | NA | NA | 0.476 |
| A_24_P47681 | NM_018448 | CAND1 | cullin-associated | TBP-interacting | 0.474 |
| | | | and neddylation- | protein | |
| | | | dissociated 1 | | |
| A_24_P213073 | NA | NA | NA | NA | 0.474 |
| A_24_P384411 | NA | NA | NA | NA | 0.474 |
| A_24_P41149 | NA | NA | NA | NA | 0.471 |
| A_24_P780052 | NM_001005472 | NA | NA | NA | 0.471 |
| A_32_P105940 | NA | NA | NA | NA | 0.468 |
| A_23_P357717 | NM_021966 | TCL1A | T-cell | T-cell | 0.468 |
| | | | leukemia/lymphoma | leukemia/lymphoma | |
| | | | 1A | 1A | |
| A_24_P31165 | NM_002055 | GFAP | glial fibrillary acidic | glial fibrillary acidic | 0.465 |
| | | | protein | protein | |
| A_24_P169645 | NA | NA | NA | NA | 0.464 |
| A_23_P138125 | NM_005449 | FAIM3 | Fas apoptotic | interleukin 24 | 0.463 |
| | | | inhibitory molecule 3 | | |
| A_24_P375405 | NA | NA | NA | NA | 0.462 |
| A_24_P341006 | XR_015921 | NA | NA | NA | 0.462 |
| A_23_P31376 | NM_018334 | LRRN3 | leucine rich repeat | leucine rich repeat | 0.460 |
| | | | neuronal 3 | neuronal 3 | |
| A_24_P272403 | BE816155 | NA | NA | NA | 0.459 |
| A_24_P178654 | XR_018292 | NA | NA | NA | 0.459 |
| A_24_P349596 | XR_018451 | NA | NA | NA | 0.458 |
| A_32_P157631 | NA | NA | NA | NA | 0.456 |
| A_24_P383802 | XR_019516 | NA | NA | NA | 0.456 |
| A_32_P186038 | NA | NA | NA | NA | 0.455 |
| A_24_P307025 | NR_000029 | RPL23AP7 | ribosomal protein | NA | 0.452 |
| | | | L23a pseudogene 7 | | |
| A_24_P238427 | NA | NA | NA | NA | 0.452 |
| A_24_P505981 | NA | NA | NA | NA | 0.450 |
| A_24_P940348 | NM_173544 | FAM129C | family with | B-cell novel protein 1 | 0.450 |
| | | | sequence similarity | | |
| | | | 129, member C | | |
| A_24_P807445 | NA | NA | NA | NA | 0.449 |
| A_24_P169855 | XR_016930 | NA | NA | NA | 0.447 |
| A_24_P350008 | NA | NA | NA | NA | 0.444 |
| A_24_P161317 | NA | NA | NA | NA | 0.443 |
| A_24_P40757 | XM_928198 | NA | NA | NA | 0.441 |
| A_24_P400751 | NA | NA | NA | NA | 0.438 |
| A_32_P211248 | AJ276555 | LOC100131138 | similar to | NA | 0.436 |
| | | | hCG2040918 | | |
| A_23_P59888 | NR_002182 | NACAP1 | nascent- | NA | 0.434 |
| | | | polypeptide- | | |
| | | | associated complex | | |
| | | | alpha polypeptide | | |
| | | | pseudogene 1 | | |
| A_24_P92661 | XR_019597 | NA | NA | NA | 0.433 |
| A_24_P306945 | AK090474 | LOC441245 | hypothetical | NA | 0.433 |
| | | | LOC441245 | | |
| A_32_P145856 | NA | NA | NA | NA | 0.430 |
| A_23_P315320 | NM_145659 | IL27 | interleukin 27 | interleukin 27 | 0.424 |
| A_24_P289573 | NA | NA | NA | NA | 0.413 |
| A_24_P93452 | NA | NA | NA | NA | 0.412 |
| A_24_P698816 | NA | NA | NA | NA | 0.408 |
| A_32_P334340 | AB016898 | C6orf124 | chromosome 6 | NA | 0.408 |
| | | | open reading frame | | |
| | | | 124 | | |
| A_24_P392271 | NA | NA | NA | NA | 0.408 |
| A_24_P237328 | NM_014507 | MCAT | malonyl CoA:ACP | malonyl-CoA:acyl | 0.405 |
| | | | acyltransferase | carrier protein | |
| | | | (mitochondrial) | transacylase, | |
| | | | | mitochondrial | |
| A_24_P418189 | XR_018242 | NA | NA | NA | 0.403 |
| A_24_P412734 | NM_173502 | PRSS36 | protease, serine, 36 | protease, serine, 36 | 0.401 |
| A_24_P76120 | NA | NA | NA | NA | 0.395 |
| A_24_P101211 | XR_018768 | NA | NA | NA | 0.387 |
| A_24_P307443 | XR_018808 | NA | NA | NA | 0.386 |
| A_24_P366768 | XR_018308 | NA | NA | NA | 0.386 |
| A_24_P272735 | NA | NA | NA | NA | 0.385 |
| A_24_P126902 | NA | NA | NA | NA | 0.383 |
| A_24_P456884 | BC047952 | LOC100130890 | similar to | NA | 0.379 |
| | | | hCG2030844 | | |
| A_24_P195556 | XR_019603 | NA | NA | NA | 0.373 |
| A_24_P195510 | XR_019574 | NA | NA | NA | 0.362 |
| A_24_P323635 | XM_070233 | NA | NA | NA | 0.359 |
| A_24_P204165 | NA | NA | NA | NA | 0.345 |
| A_24_P379649 | NR_002229 | RPL23AP32 | ribosomal protein | NA | 0.339 |
| | | | L23a pseudogene | | |
| | | | 32 | | |
| A_24_P417352 | BX161420 | IGHM | immunoglobulin | NA | 0.318 |
| | | | heavy constant mu | | |
| A_24_P41662 | NA | NA | NA | NA | 0.279 |

1. A method of diagnosing interstitial lung disease in a subject or identifying a subject having an increased risk of developing interstitial lung disease, comprising:
a. analyzing at least one biomarker in a sample from the subject; and
b. comparing the analysis of (a) with an analysis of the at least one biomarker in individual samples from a group of mild interstitial lung disease subjects and/or a group of severe interstitial lung disease subjects,
wherein an analysis of (a) that is similar to the analysis of (b) diagnoses interstitial lung disease in the subject or identifies the subject as having an increased risk of developing interstitial lung disease.

2. A method of diagnosing interstitial lung disease in a subject or identifying a subject having an increased risk of developing interstitial lung disease, comprising:
a. analyzing at least one biomarker in a sample from the subject; and
b. comparing the analysis of (a) with an analysis of the at least one biomarker in individual samples from a group of control subjects,
wherein an analysis of (a) that is different than the analysis of (b) diagnoses interstitial lung disease in the subject or identifies the subject as having an increased risk of developing interstitial lung disease.

3. (canceled)

4. (canceled)

5. (canceled)

6. A method of diagnosing interstitial lung disease in a subject or identifying a subject as having an increased risk of developing interstitial lung disease, comprising:
a. quantifying the amount of at least one biomarker in a sample from the subject;
b. comparing the amount of the at least one biomarker quantified in (a) with the amount of the at least one biomarker quantified in individual samples from a group of mild interstitial lung disease subjects and/or a group of severe interstitial lung disease subjects; and
c. diagnosing interstitial lung disease in the subject or identifying the subject as having an increased risk of developing interstitial lung disease based on the comparison of the amount of the at least one biomarker of steps (a) and (b).

7. A method of diagnosing interstitial lung disease in a subject or identifying a subject as having an increased risk of developing interstitial lung disease, comprising:
a. quantifying the amount of at least one biomarker in a sample from the subject;
b. comparing the amount of the at least one biomarker quantified in (a) with the amount of the at least one biomarker quantified in individual samples from a group of control subjects; and
c. diagnosing interstitial lung disease in the subject or identifying the subject as having an increased risk of developing interstitial lung disease based on the comparison of the amount of the at least one biomarker of steps (a) and (b).

8. (canceled)

9. (canceled)

10. (canceled)

11. The method of claim 1, wherein the interstitial lung disease is idiopathic interstitial pneumonia (IIP).

12. The method of claim 11, wherein the IIP is familial interstitial pneumonia (FIP).

13. The method of claim 1, wherein the biomarker is selected from the group consisting of the biomarkers of Table 2, the biomarkers of Table 3, the biomarkers of Table 4, the biomarkers of Table 5, the biomarkers of Table 12, the biomarkers of Table 13 and any combination thereof.

14. A method of identifying a subject having an increased risk of developing severe interstitial lung disease, comprising:
a. analyzing at least one biomarker in a sample from the subject; and
b. comparing the analysis of (a) with an analysis of the at least one biomarker in samples from a group of control subjects,
wherein an analysis of (a) that is different than the analysis of (b) identifies the subject as having an increased risk of developing severe interstitial lung disease.

15. A method of identifying a subject as having an increased risk of developing severe interstitial lung disease, comprising:
a. quantifying the amount of at least one biomarker in a sample from the subject;
b. comparing the amount of the at least one biomarker quantified in (a) with the amount of the at least one biomarker quantified in samples from a group of control subjects; and
c. identifying the subject as having an increased risk of developing severe interstitial lung disease based on the comparison of the amount of the at least one biomarker of steps (a) and (b).

16. The method of claim 14, wherein the subject has mild interstitial lung disease.

17. The method of claim 14, wherein the biomarker is selected from the group consisting of CAMP, CEACAM6, CTSG, DEFA3, DEFA4, OLFM4, HLTF and any combination thereof and wherein the analysis of (a) that is different than the analysis of (b) is an increase in an amount of the at least one biomarker in the sample from the subject relative to an amount of the at least one biomarker in the samples from the group of control subjects.

18. The method of claim 15, wherein the biomarker is selected from the group consisting of CAMP, CEACAM6, CTSG, DEFA3, DEFA4, OLFM4, HLTF and any combination thereof and wherein the comparison of the amount of the at least one biomarker of steps (a) and (b) shows an increase in an amount of the at least one biomarker of step (a) relative to an amount of the at least one biomarker of step (b).

19. The method of claim 14, wherein the biomarker is selected from the group consisting of PACSIN1, FLJ11710, GABBR1, IGHM and any combination thereof, and the analysis of (a) that is different than the analysis of (b) is a decrease in an amount of the at least one biomarker in the sample from the subject relative to an amount of the at least one biomarker in the samples from the group of control subjects.

20. The method of claim 15, wherein the biomarker is selected from the group consisting of PACSIN1, FLJ11710, GABBR1, IGHM and any combination thereof and wherein the comparison of the amount of the at least one biomarker of steps (a) and (b) shows a decrease in an amount of the at least one biomarker of step (a) relative to an amount of the at least one biomarker of step (b).

21. The method of claim 1, wherein the sample is selected from the group consisting of blood, bronchoalveolar lavage fluid, plasma, serum, sputum, tissue, cells and any combination thereof.

22-29. (canceled)
